(19) United States
(12) Patent Application Publication
(10) Pub. No.: US 2024/0238346 A1
(43) Pub. Date: Jul. 18, 2024

(54) MYOCARDIAL WOUND HEALING POST ISCHEMIC INJURY

(71) Applicant: Temple University - of the Commonwealth System of Higher Education, Philadelphia, PA (US)

(72) Inventors: Sadia MOHSIN, Philadelphia, PA (US); Steven HOUSER, Philadelphia, PA (US); Hajime KUBO, Philadelphia, PA (US)

(21) Appl. No.: 18/577,300

(22) PCT Filed: Jul. 8, 2022
(86) PCT No.: PCT/US22/36457

§ 371 Date: Jan. 8, 2024

Related U.S. Application Data
(60) Provisional application No. 63/220,235, filed on Jul. 9, 2021.

(51) Int. Cl. A61K 35/28; A61K 38/17; A61P 9/00

(52) U.S. Cl. CPC A61K 35/28; A61K 38/1754; A61K 38/1793; A61P 9/00

(57) ABSTRACT

Cell therapies and cell secretome compositions are utilized in the methods of treatment of cardiac injury. The compositions mediate myocardial homeostasis and cardiac wound healing process post-MI via the direct modulation of regulatory T cell (Treg) population dynamics and function.

### CROSS REFERENCE TO RELATED APPLICATIONS

This application claims the benefit of U.S. Provisional Application 63/220,235 filed on Jul. 9, 2021. The entire contents of this application are incorporated herein by reference in their entirety.

### STATEMENT REGARDING FEDERALLY SPONSORED RESEARCH

This invention was made with government support under Grant Numbers 361499 and HL137850 awarded by the National Institutes of Health. The government has certain rights in the invention.

### FIELD

Cell therapies mediate myocardial homeostasis and cardiac wound healing process post-myocardial infraction (MI) via the direct modulation of regulatory T cell (Treg) population dynamics and function.

### BACKGROUND

Myocardial Infarction (MI) causes cardiomyocyte death, which compromises the integrity of the myocardium and results in adverse cardiac remodeling, compromised heart function, and heart failure pathology. Cell-based therapies were designed with the intent that the adoptive transfer of progenitor cell populations into the infarcted heart would differentiate into new, functional myocardium to replace the tissue lost during MI.¹ Unfortunately, multiple pre-clinical and clinical trials using stem cell therapies identified that the transdifferentiation of progenitor stem cells, regardless of cell type, is a rare occurrence and not the main reparative processes that mediates the cardiogenic capacity of cell therapy; 1-3 but rather the paracrine signaling intermediates produced by progenitor populations are an ideal therapeutic target to solicit stem cell mediated cardiac repair.⁴⁻⁶

Following MI, cardiac repair processes must facilitate the clearance of necrotic myocardium and the promotion of wound healing processes that aim to revascularize and strengthen the ischemic myocardial wall to prevent cardiac rupture. The mounted inflammatory response is closely coupled to myocardial repair following sterile, ischemic injury via a multitude of inflammatory cell types possessed within both the innate and adaptive immune responses.^{7,8}

The exact role T-lymphocytes serve in modulating cardiac repair is variable and highly dependent upon the T-cell subset population of investigation. In general, CD3⁺ T-cell populations can be further characterized into CD4⁺ T helper cell populations or CD8⁺ cytotoxic T-cells, both of which have been shown to negatively contribute to cardiac wound healing processes.⁹⁻¹¹ However, a smaller subset of CD4⁺ T-cells retaining FoxP3 transcription factor expression are referred to as T-regulatory Cells (Tregs).¹² Tregs are traditionally described as the main immunosuppressive cell type in the peripheral immune system and are responsible for dampening other T cell subset responses.¹³⁻¹⁵ However, recent reports identify Treg cells possess extra-immunological processes that can maintain tissue homeostasis, earning the designation of Tissue Tregs.¹⁶ Recently, a cardiac specific Treg population that responds to ischemic injury (MI and IR) has been identified.¹⁷ Cardiac Treg populations are phenotypically distinct from other Treg populations that occupy the skeletal muscle, skin, and peripheral lymphoid organs. The ablation of these populations or the blockage of their recruitment to the MI heart compromises cardiac wound healing processes, identifying the necessity these populations have in orchestrating myocardial repair post-MI.¹⁷ In contradiction, a previous report identified that Tregs undergo phenotypic, malfunctional conversions during chronic ischemic injury, resulting in pathogenic, TNFRI⁺ Treg establishment that results in adverse cardiac remodeling and function.¹⁸ Identifying a therapeutic that can modulate the expansion and sustainment of pro-reparative, TNFRII⁺ Tregs in the ischemic heart can greatly improve cardiac wound healing processes.

### SUMMARY

Therapies comprising cortical bone derived stem cells (CBSCs) mediate myocardial homeostasis and cardiac wound healing process post-MI via the direct modulation of regulatory T cell (Treg) population dynamics and function are provided herein.

Accordingly, in certain embodiments, a composition comprises a therapeutically effective amount of cortical bone derived stem cells (CBSCs) and/or CBSC secretome.

In certain embodiments, a method of promoting myocardial homeostasis and cardiac wound healing in a subject in need thereof, comprises administering to the subject a therapeutically effective amount of cortical bone derived stem cells (CBSCs); thereby, promoting myocardial homeostasis and cardiac wound healing in a subject. In certain embodiments, the CBSCs are administered via intramyocardial injection. In certain embodiments, the CBSCs are autologous, allogeneic, haplotype matched, haplotype mismatched, haplo-identical, xenogeneic or combinations thereof. In certain embodiments, the method further comprises administering a therapeutically effective amount of a CBSC secretome. In certain embodiments, the CBSC secretome comprises osteoprotegrin (OPG) and/or insulin-like growth factor-binding protein 5 (IGFBP-5). In certain embodiments, the CBSCs and/or secretome induce T regulatory (Treg) cells. In certain embodiments, the CBSCs and/or secretome mediate recruitment and expansion of Treg from peripheral T cell stores into the subject's infarcted heart. In certain embodiments, the CBSCs and/or secretome modulate the inflammatory microenvironment of the subject's infarcted heart. In certain embodiments, the induced Treg cells are TNFRII+. In certain embodiments, the CBSCs are expanded ex vivo. In certain embodiments, the CBSC secretome is enriched ex vivo by culturing the CBSCs for at least 12 hours. In certain embodiments, the method further comprises culturing Treg cells ex vivo with the CBSC secretome. In certain embodiments, the Treg cells are TNFRII+. In certain embodiments, the TNFRII+ Treg cells are adoptively transferred to the subject.

### Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The term “cardiomyocyte” as used herein broadly refers to a muscle cell of the heart. The term cardiomyocyte includes cardiac muscle cells, which include also include striated muscle cells, as well as spontaneous beating muscle cells of the heart.

As used herein, the phrase “cardiovascular condition, disease or disorder” is intended to include all disorders characterized by insufficient, undesired or abnormal cardiac function, e.g. ischemic heart disease, hypertensive heart disease and pulmonary hypertensive heart disease, valvular disease, congenital heart disease and any condition which leads to congestive heart failure in a subject, particularly a human subject. Insufficient or abnormal cardiac function can be the result of disease, injury and/or aging. By way of background, a response to myocardial injury follows a well-defined path in which some cells die while others enter a state of hibernation where they are not yet dead but are dysfunctional. This is followed by infiltration of inflammatory cells, deposition of collagen as part of scarring, all of which happen in parallel with in-growth of new blood vessels and a degree of continued cell death.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

The terms “coronary artery disease” and “acute coronary syndrome” as used interchangeably herein, and refer to myocardial infarction refer to a cardiovascular condition, disease or disorder, include all disorders characterized by insufficient, undesired or abnormal cardiac function, e.g. ischemic heart disease, hypertensive heart disease and pulmonary hypertensive heart disease, valvular disease, congenital heart disease and any condition which leads to congestive heart failure in a subject, particularly a human subject. Insufficient or abnormal cardiac function can be the result of disease, injury and/or aging. By way of background, a response to myocardial injury follows a well-defined path in which some cells die while others enter a state of hibernation where they are not yet dead but are dysfunctional. This is followed by infiltration of inflammatory cells, deposition of collagen as part of scarring, all of which happen in parallel with in-growth of new blood vessels and a degree of continued cell death.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The term “effective amount” as used herein refers to the amount of therapeutic agent of pharmaceutical composition, e.g., an amount of osteoprotegrin (OPG) and/or insulin-like growth factor-binding protein 5 (IGFBP-5) to reduce at least one or more symptom(s) of the disease or disorder and relates to a sufficient amount of pharmacological composition to provide the desired effect. The phrase “therapeutically effective amount” as used herein, e.g., of CBSCs and/or CBSC secretome or secretome compositions as disclosed herein means a sufficient amount of the composition to treat a disorder, at a reasonable benefit/risk ratio applicable to any medical treatment. The term “therapeutically effective amount” therefore refers to an amount of the composition as disclosed herein that is sufficient to, for example, effect a therapeutically or prophylactically significant reduction in a symptom or clinical marker associated with a cardiac dysfunction or disorder when administered to a typical subject who has a cardiovascular condition, disease or disorder.

With reference to the treatment of, for example, a cardiovascular condition or disease in a subject, the term “therapeutically effective amount” refers to the amount that is safe and sufficient to prevent or delay the development or a cardiovascular disease or disorder. The amount can thus cure or cause the cardiovascular disease or disorder to go into remission, slow the course of cardiovascular disease progression, slow or inhibit a symptom of a cardiovascular disease or disorder, slow or inhibit the establishment of secondary symptoms of a cardiovascular disease or disorder or inhibit the development of a secondary symptom of a cardiovascular disease or in disorder. The effective amount for the treatment of the cardiovascular disease or disorder depends on the type of cardiovascular disease to be treated, the severity of the symptoms, the subject being treated, the age and general condition of the subject, the mode of administration and so forth. Thus, it is not possible to specify the exact “effective amount”. However, for any given case, an appropriate “effective amount” can be determined by one of ordinary skill in the art using only routine experimentation. The efficacy of treatment can be judged by an ordinarily skilled practitioner, for example, efficacy can be assessed in animal models of a cardiovascular disease or disorder as discussed herein, for example treatment of a rodent with acute myocardial infarction or ischemia-reperfusion injury, and any treatment or administration of the compositions or formulations that leads to a decrease of at least one symptom of the cardiovascular disease or disorder as disclosed herein, for example, increased heart ejection fraction, decreased rate of heart failure, decreased infarct size, decreased associated morbidity (pulmonary edema, renal failure, arrhythmias) improved exercise tolerance or other quality of life measures, and decreased mortality indicates effective treatment. In embodiments where the compositions are used for the treatment of a cardiovascular disease or disorder, the efficacy of the composition can be judged using an experimental animal model of cardiovascular disease, e.g., as explained in detail in the examples section, or for example, animal models of ischemia-reperfusion injury (Headrick J P, Am J Physiol Heart Circ Physiol 285; H1797; 2003) and animal models acute myocardial infarction. (Yang Z, Am J Physiol Heart Circ. Physiol 282:H949: 2002; Guo Y, J Mol Cell Cardiol 33;825-830, 2001). When using an experimental animal model, efficacy of treatment is evidenced when a reduction in a symptom of the cardiovascular disease or disorder, for example, a reduction in one or more symptom of dyspnea, chest pain, palpitations, dizziness, syncope, edema, cyanosis, pallor, fatigue, and high blood pressure which occurs earlier in treated, versus untreated animals.

Subjects amenable to treatment by the methods as disclosed herein can be identified by any method to diagnose myocardial infarction (commonly referred to as a heart attack). Methods of diagnosing these conditions are well known by persons of ordinary skill in the art. By way of non-limiting example, myocardial infarction can be diagnosed by (i) blood tests to detect levels of creatine phosphokinase (CPK), aspartate aminotransferase (AST), lactate dehydrogenase (LDH) and other enzymes released during myocardial infarction; (ii) electrocardiogram (ECG or EKG) which is a graphic recordation of cardiac activity, either on paper or a computer monitor. An ECG can be beneficial in detecting disease and/or damage; (iii) echocardiogram (heart ultrasound) used to investigate congenital heart disease and assessing abnormalities of the heart wall, including functional abnormalities of the heart wall, valves and blood vessels; (iv) Doppler ultrasound can be used to measure blood flow across a heart valve; (v) nuclear medicine imaging (also referred to as radionuclide scanning in the art) allows visualization of the anatomy and function of an organ, and can be used to detect coronary artery disease, myocardial infarction, valve disease, heart transplant rejection, check the effectiveness of bypass surgery, or to select patients for angioplasty or coronary bypass graft.

The term “enhancement,” “enhance,” “enhances,” or “enhancing” refers to an increase in the specified parameter (e.g., at least about a 1.1-fold, 1.25-fold, 1.5-fold, 2-fold, 3-fold, 4-fold, 5-fold, 6-fold, 8-fold, 10-fold, twelve-fold, or even fifteen-fold or more increase) and/or an increase in the specified activity of at least about 5%, 10%, 25%, 35%, 40%, 50%, 60%, 75%, 80%, 90%, 95%, 97%, 98%, 99% or 100%.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The term “inhibit,” “diminish,” “reduce” or “suppress” refers to a decrease in the specified parameter (e.g., at least about a 1.1-fold, 1.25-fold, 1.5-fold, 2-fold, 3-fold, 4-fold, 5-fold, 6-fold, 8-fold, 10-fold, twelve-fold, or even fifteen-fold or more increase) and/or a decrease or reduction in the specified activity of at least about 5%, 10%, 25%, 35%, 40%, 50%, 60%, 75%, 80%, 90%, 95%, 97%, 98%, 99% or 100%. These terms are intended to be relative to a reference or control.

As used herein, the term “ischemia” refers to any localized tissue ischemia due to reduction of the inflow of blood. The term “myocardial ischemia” refers to circulatory disturbances caused by coronary atherosclerosis and/or inadequate oxygen supply to the myocardium. For example, an acute myocardial infarction represents an irreversible ischemic insult to myocardial tissue. This insult results in an occlusive (e.g., thrombotic or embolic) event in the coronary circulation and produces an environment in which the myocardial metabolic demands exceed the supply of oxygen to the myocardial tissue.

As used herein, “modulate,” “modulates” or “modulation” refers to enhancement (e.g., an increase) or inhibition (e.g., diminished, reduced or suppressed) of the specified activity or expression of a gene, polynucleotides, oligonucleotides, proteins, polypeptides, peptides or combinations thereof.

Unless specifically stated or obvious from context, as used herein, the term “or” is understood to be inclusive.

The terms “patient” or “individual” or “subject” are used interchangeably herein, and refers to a mammalian subject to be treated, with human patients being preferred. In some cases, the methods of the disclosure find use in experimental animals, in veterinary application, and in the development of animal models for disease, including, but not limited to, rodents including mice, rats, and hamsters, and primates. The terms include any animal that has, or is suspected of having, a myocardial injury, for example, myocardial ischemia. Suitable subjects (patients) include laboratory animals (such as mouse, rat, rabbit, guinea pig or pig), farm animals, sporting animals (e.g., dogs or horses) and domestic animals or pets (such as a horse, dog or cat). Non-human primates and human patients are included. For example, human subjects who present with chest pain or other symptoms of cardiac distress, including, e.g., shortness of breath, nausea, vomiting, sweating, weakness, fatigue, or palpitations, can be evaluated by a method of the invention. About ¼ of myocardial infarction (MI) are silent and without chest pain. Furthermore, patients who have been evaluated in an emergency room or in an ambulance or physician's office and then dismissed as not being ill according to current tests for infarction have an increased risk of having a heart attack in the next 24-48 hours; such patients can be monitored by a method of the invention to determine if and when they begin express markers of the invention, which indicates that, e.g., they are beginning to exhibit ischemia. Subjects can also be monitored by a method of the invention to improve the accuracy of current provocative tests for ischemia, such as exercise stress testing. An individual can be monitored by a method of the invention during exercise stress tests or Dobutamine stress tests to determine if the individual is at risk for ischemia; such monitoring can supplement or replace the test that is currently carried out. Athletes (e.g., humans, racing dogs or racehorses) can be monitored during training to ascertain if they are exerting themselves too vigorously and are in danger of undergoing an MI.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The terms “sample,” “patient sample,” “biological sample,” and the like, encompass a variety of sample types obtained from a patient, individual, or subject and can be used in a diagnostic, prognostic and/or monitoring assay. The patient sample may be obtained from a healthy subject, a diseased patient or a patient having associated symptoms of cardiovascular diseases or disorders.

Various methodologies of the instant invention include a step that involves comparing a value, level, feature, characteristic, property, etc. to a “suitable control,” referred to interchangeably herein as an “appropriate control,” a “control sample,” a “reference” or simply a “control.” A “suitable control,” “appropriate control,” “control sample,” “reference” or a “control” is any control or standard familiar to one of ordinary skill in the art useful for comparison purposes. A “reference level” of a biomarker means a level of the biomarker that is indicative of a particular disease state, phenotype, or lack thereof, as well as combinations of disease states, phenotypes, or lack thereof. A “positive” reference level of a biomarker means a level that is indicative of a particular disease state or phenotype. A “negative” reference level of a biomarker means a level that is indicative of a lack of a particular disease state or phenotype. For example, a “myocardial injury-positive reference level” of a biomarker means a level of a biomarker that is indicative of a positive diagnosis of myocardial injury in a subject, and a “myocardial injury—negative reference level” of a biomarker means a level of a biomarker that is indicative of a negative diagnosis of myocardial injury in a subject. A “reference level” of a biomarker may be an absolute or relative amount or concentration of the biomarker, a presence or absence of the biomarker, a range of amount or concentration of the biomarker, a minimum and/or maximum amount or concentration of the biomarker, a mean amount or concentration of the biomarker, and/or a median amount or concentration of the biomarker; and, in addition, “reference levels” of combinations of biomarkers may also be ratios of absolute or relative amounts or concentrations of two or more biomarkers with respect to each other. Appropriate positive and negative reference levels of biomarkers for a particular disease state, phenotype, or lack thereof may be determined by measuring levels of desired biomarkers in one or more appropriate subjects, and such reference levels may be tailored to specific populations of subjects (e.g., a reference level may be age-matched so that comparisons may be made between biomarker levels in samples from subjects of a certain age and reference levels for a particular disease state, phenotype, or lack thereof in a certain age group). Such reference levels may also be tailored to specific techniques that are used to measure levels of biomarkers in biological samples (e.g., LC-MS, GC-MS, ELISA, PCR, etc.), where the levels of biomarkers may differ based on the specific technique that is used.

The term “tissue” refers to a group or layer of similarly specialized cells which together perform certain special functions. The term “tissue-specific” refers to a source or defining characteristic of cells from a specific tissue.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

The term “vector” is used to refer to a carrier nucleic acid molecule into which a heterologous nucleic acid sequence can be inserted for introduction into a cell where it can be replicated and expressed. The term further denotes certain biological vehicles useful for the same purpose, e.g. viral vectors and phage—both these infectious agents are capable of introducing a heterologous nucleic acid sequence, e.g. osteoprotegrin (OPG) and/or insulin-like growth factor-binding protein 5 (IGFBP-5).

Any compositions or methods provided herein can be combined with one or more of any of the other compositions and methods provided herein.

### DETAILED DESCRIPTION OF THE DRAWINGS

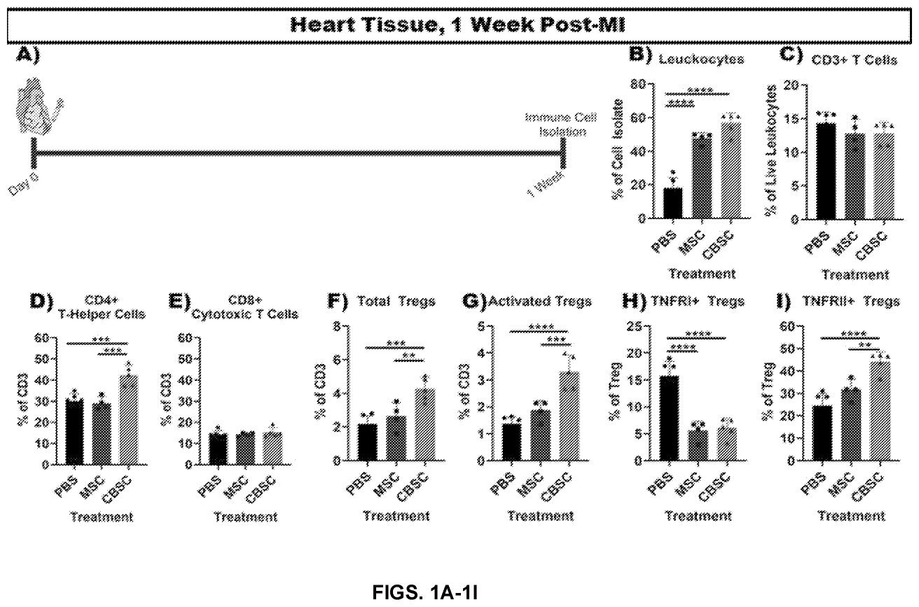

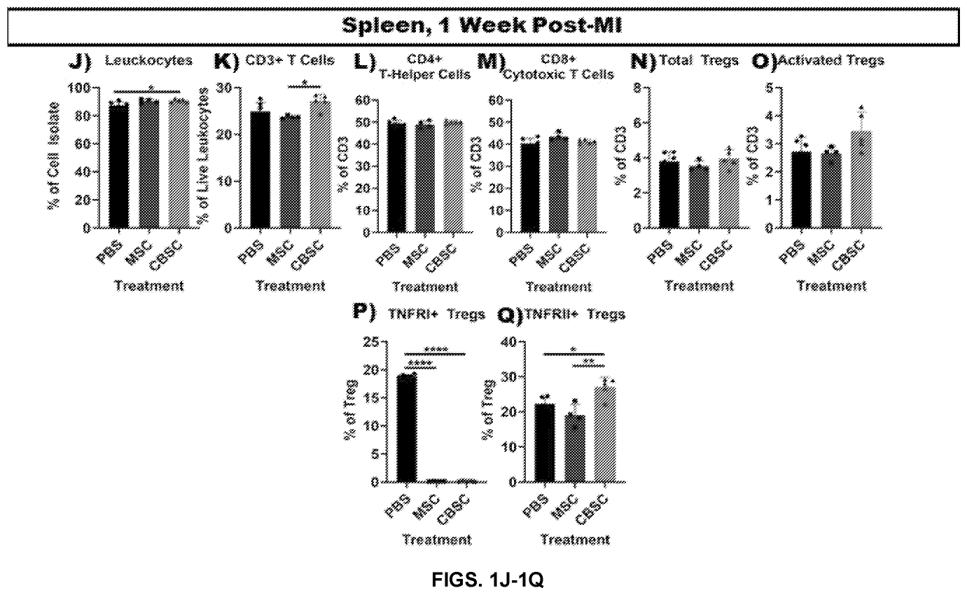

FIGS. 1A-1Q are a series of graphs and a schematic representation demonstrating that CBSC cell therapy expands an adaptive, pro-reparative inflammatory response in the injured heart and systemically 1 week post-MI.

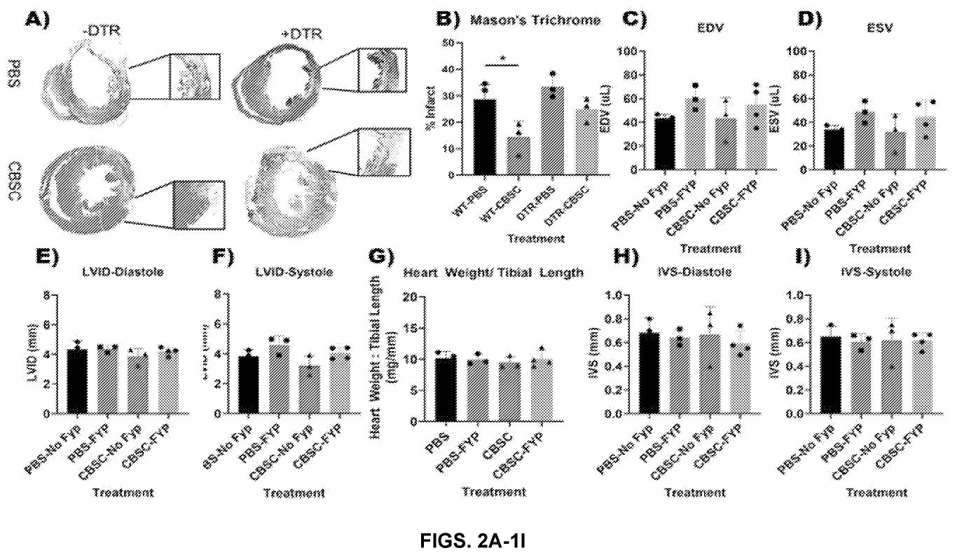

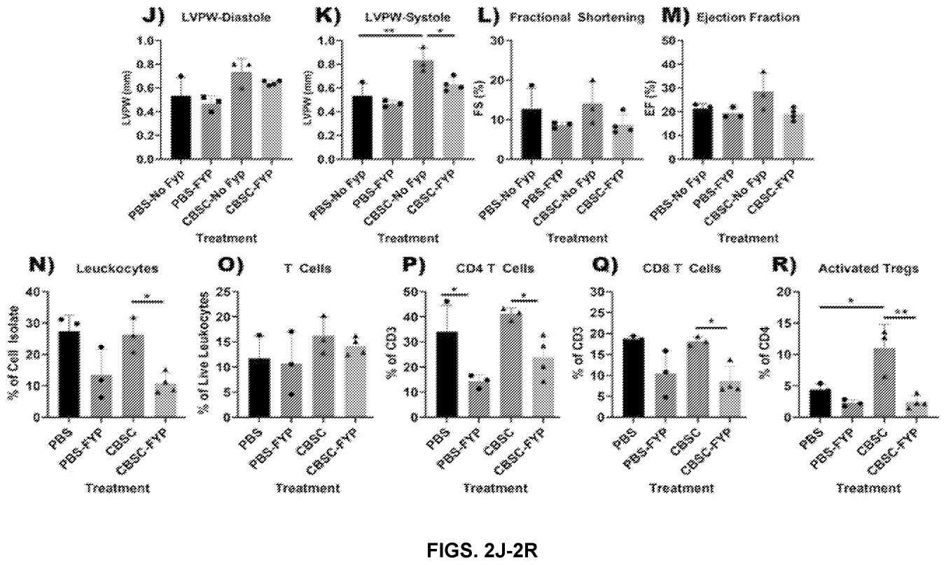

FIGS. 2A-2R are a series of tissue cross-sections and graphs demonstrating that cardiac resident Tregs post-MI are recruited to the infarcted heart from systemic lymphoid stores and are essential for CBSC mediated cardiac injury reduction post-MI.

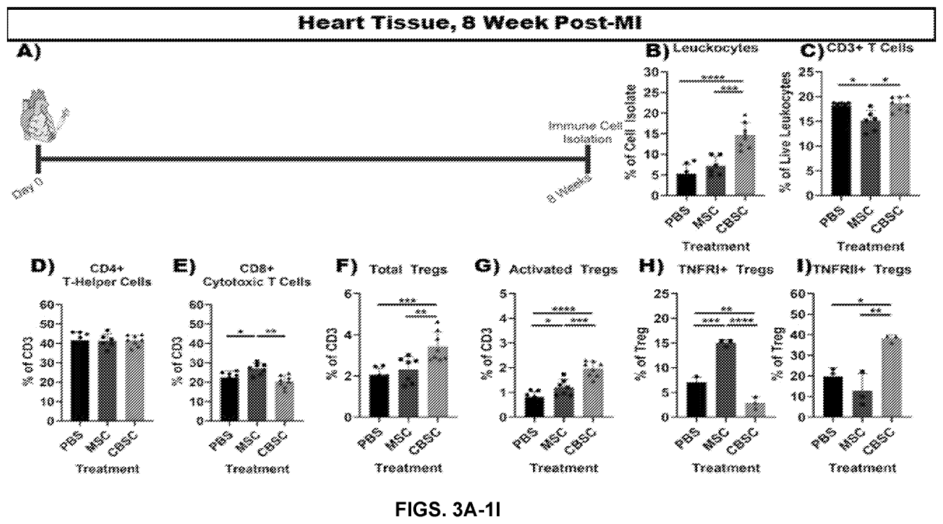

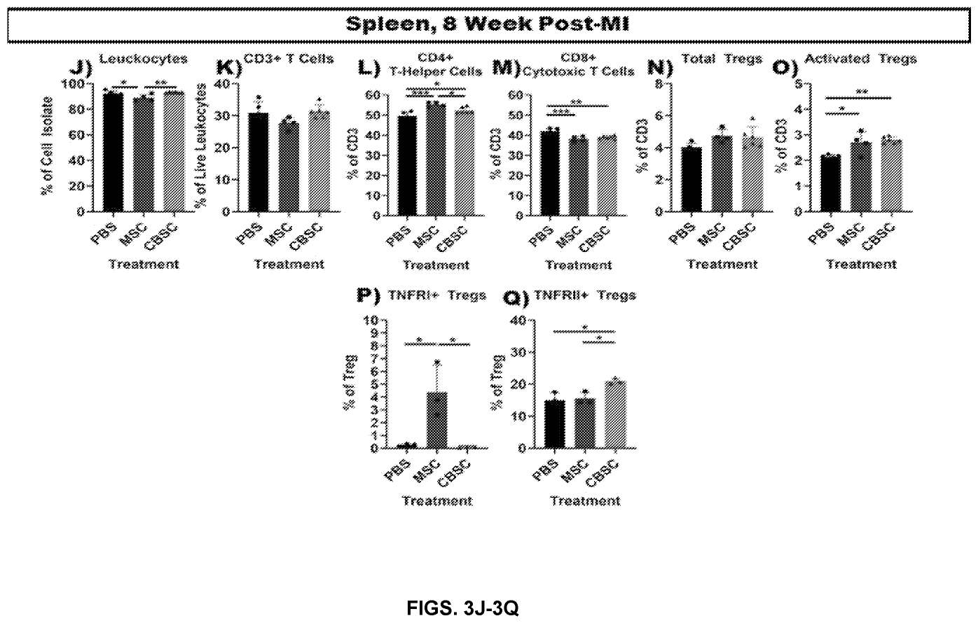

FIGS. 3A-3Q are a series of graphs and a schematic representation demonstrating that-CBSC cell therapy retains an adaptive, pro-reparative inflammatory response during chronic ischemic injury.

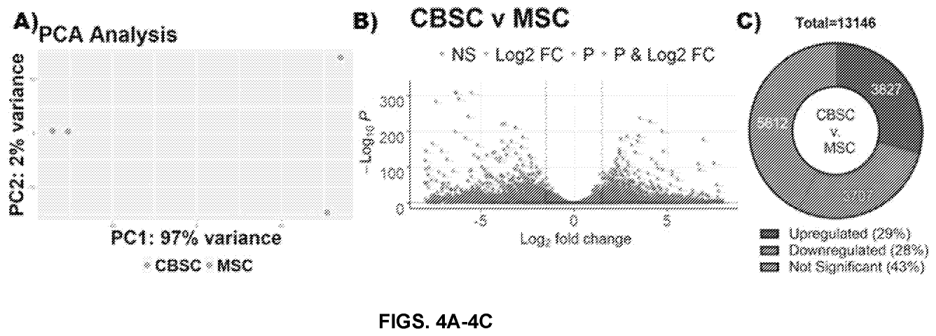

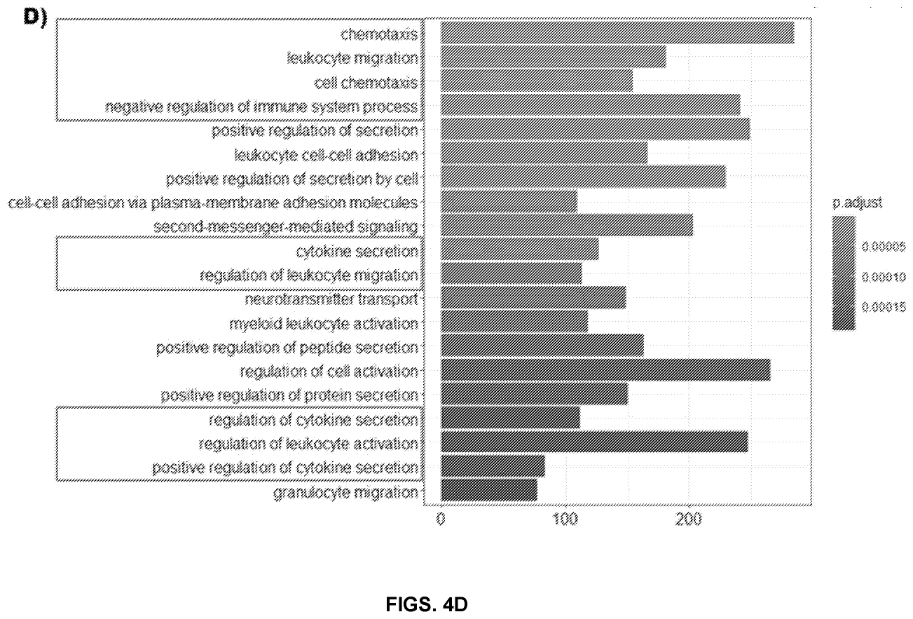

FIGS. 4A-4D are a series of plots and graphs demonstrating that CBSCs are transcriptionally distinct from MSC counterparts.

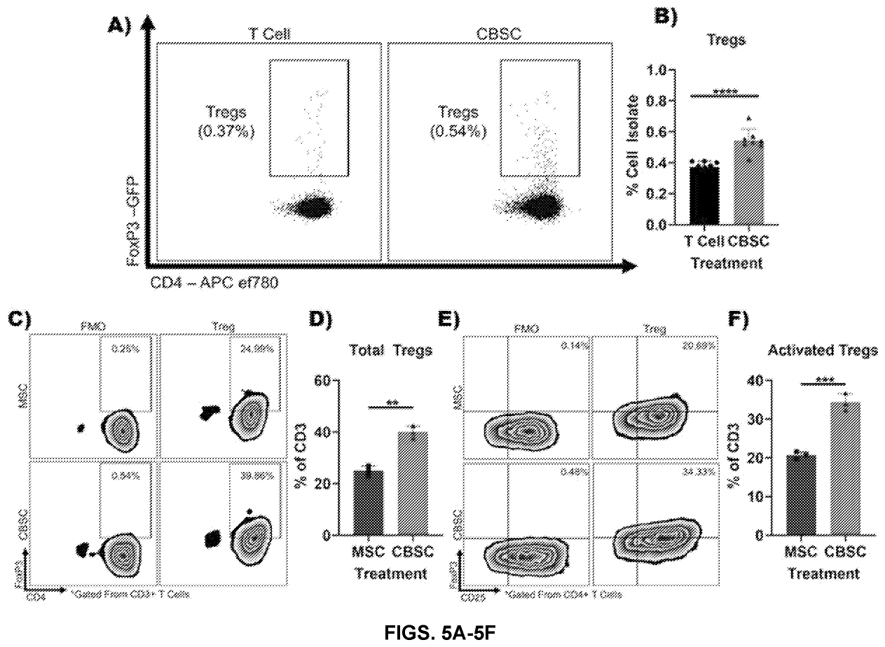

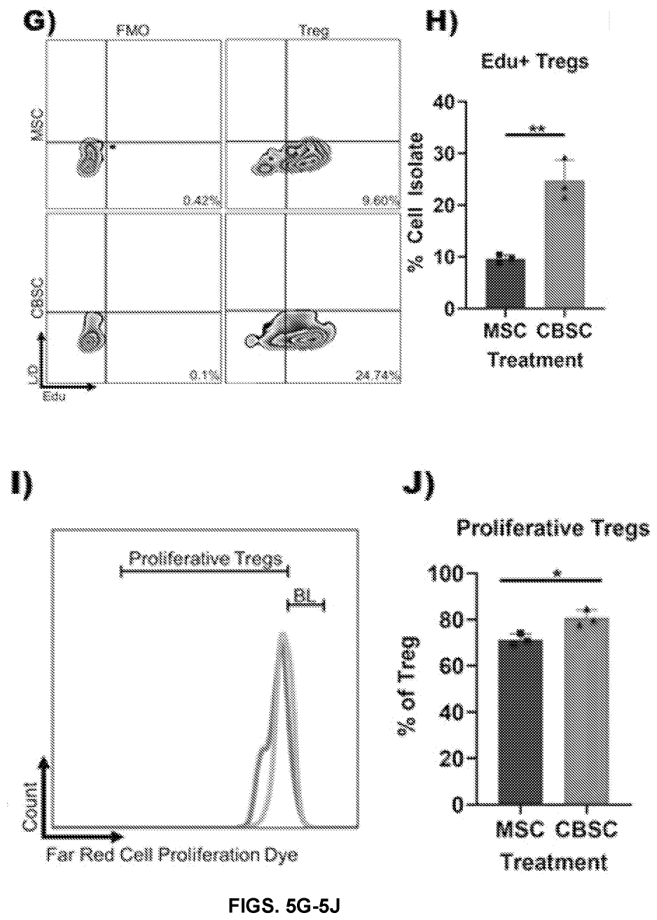

FIGS. 5A-5J are a series of plots and graphs demonstrating that the CBSC secretome directly modulates Treg cell induction and proliferation.

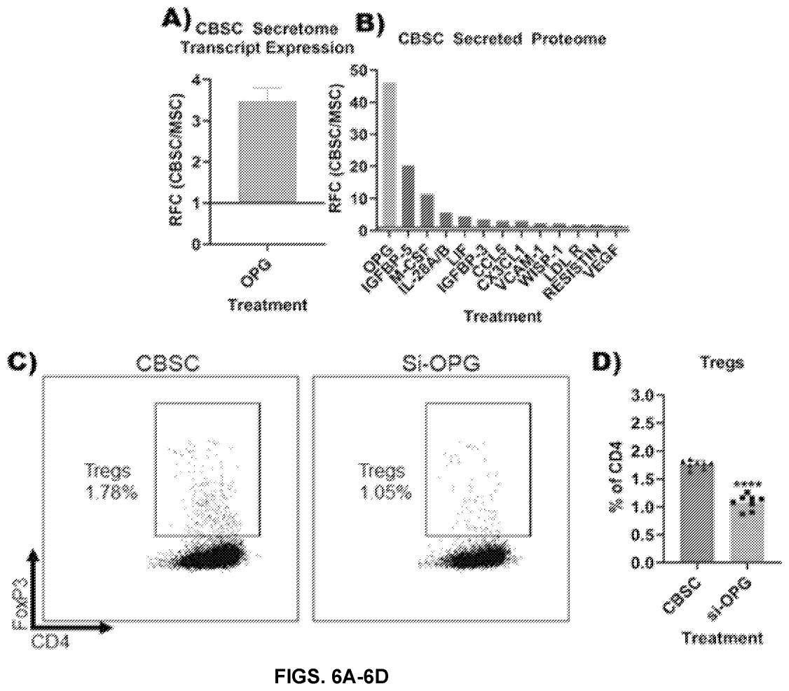

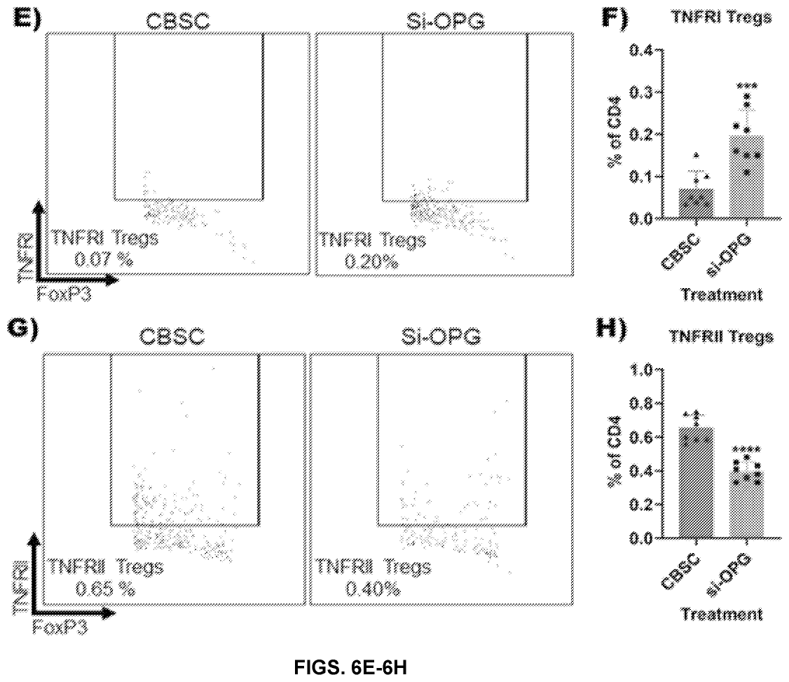

FIGS. 6A-6H are a series of plots and graphs demonstrating that CBSC produced OPG and IGFBP5 directly induces pro-reparative Treg cell phenotype induction.

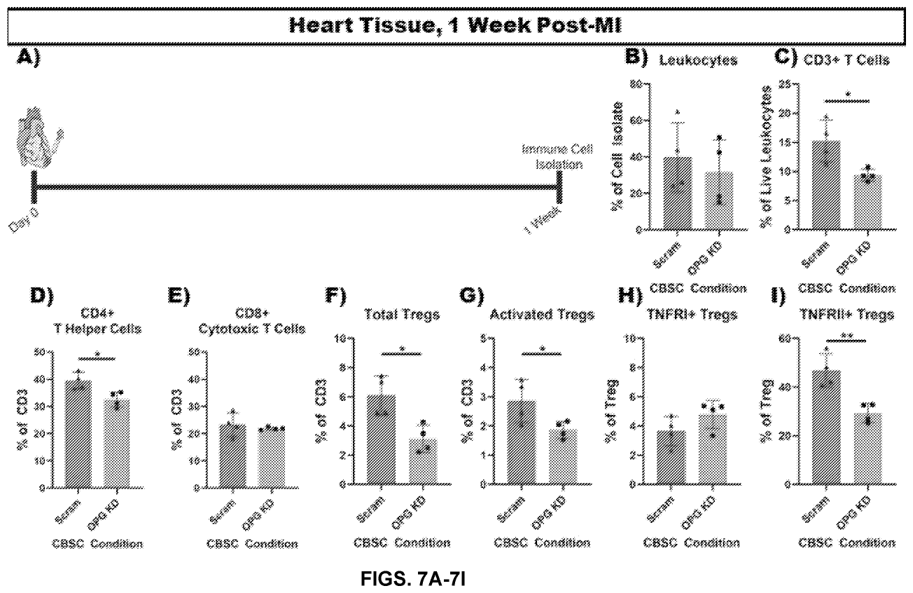

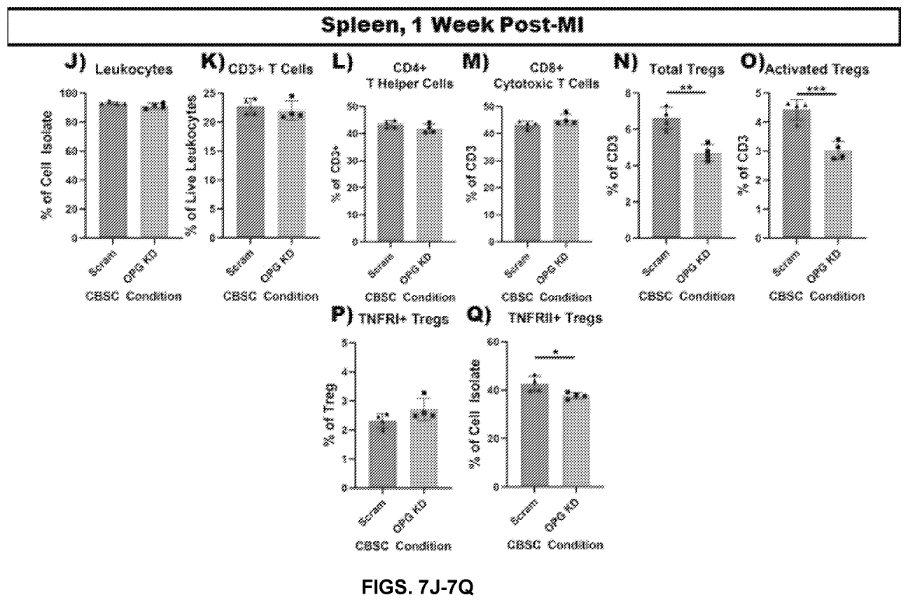

FIGS. 7A-7Q are a series of graphs and a schematic representation demonstrating that CBSC depleted of OPG compromises CBSC mediated expansion of adaptive, pro-reparative Treg response 1 week post—MI.

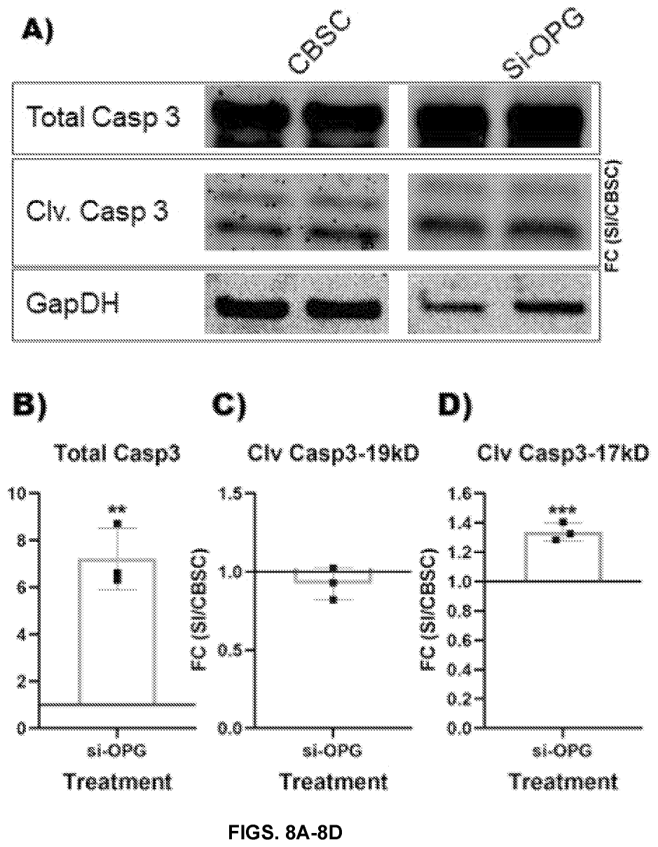

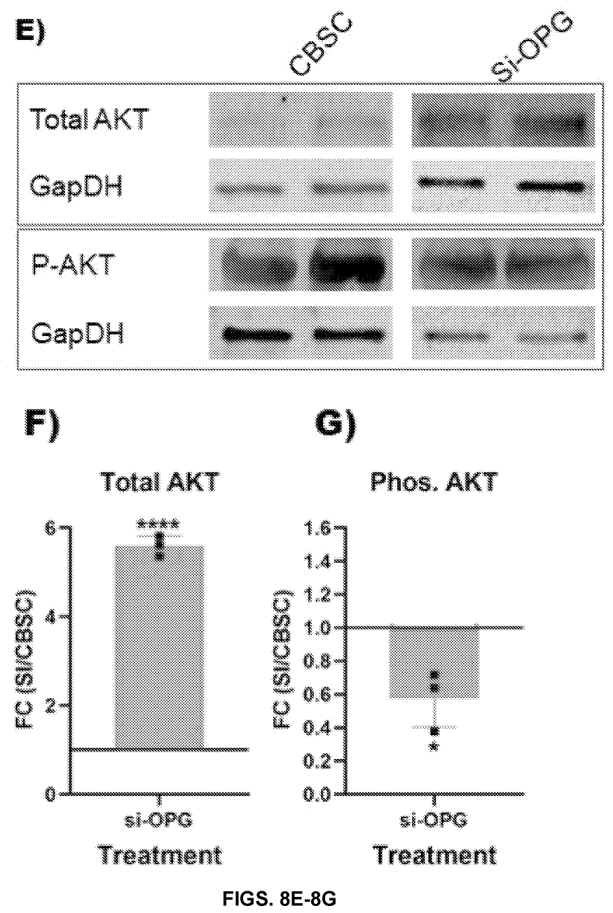

FIGS. 8A-8G are a series of blots and graphs demonstrating that CBSC produced OPG blunts T cell apoptosis and supports T cell pro-survival signaling cascades.

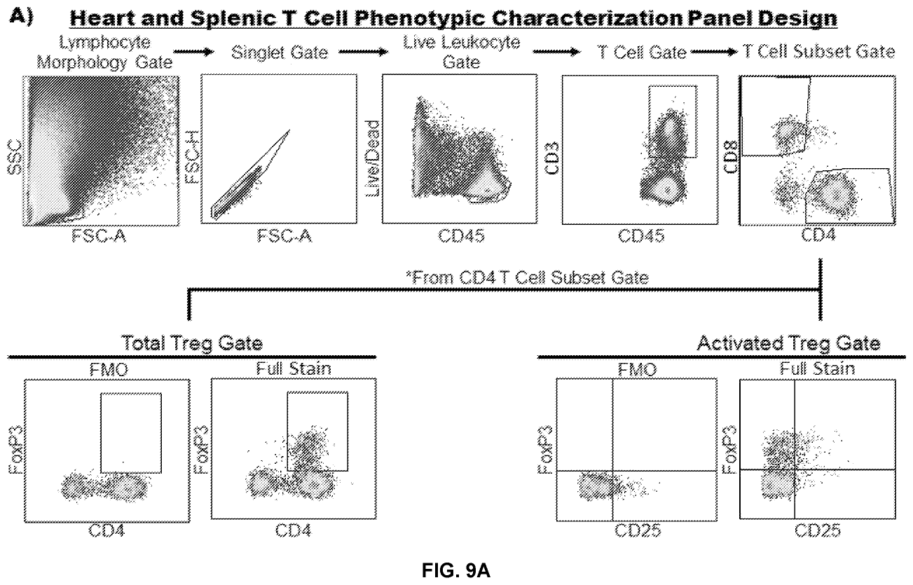

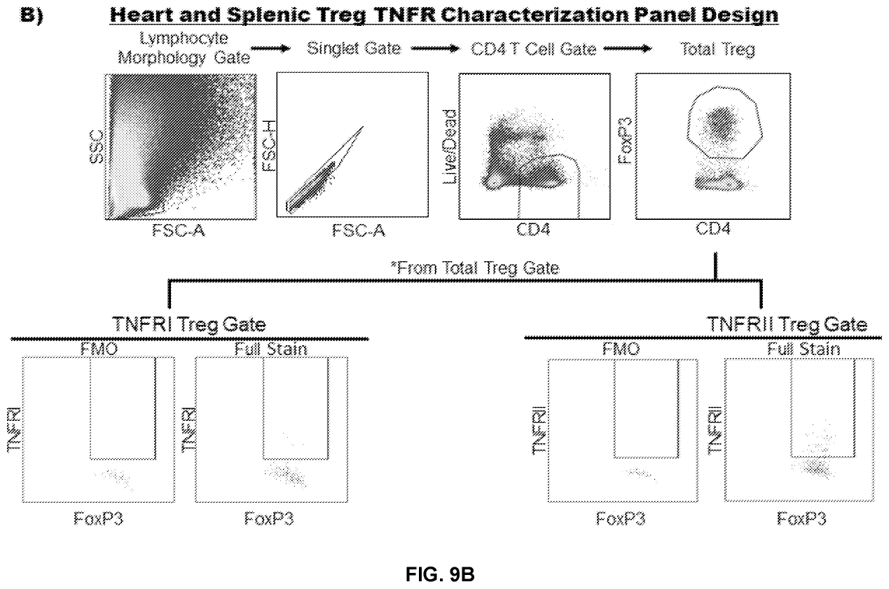

FIGS. 9A-9B are a series of plots and graphs showing the flow cytometry gating schemes used to characterize cardiac and splenic inflammatory cell composition.

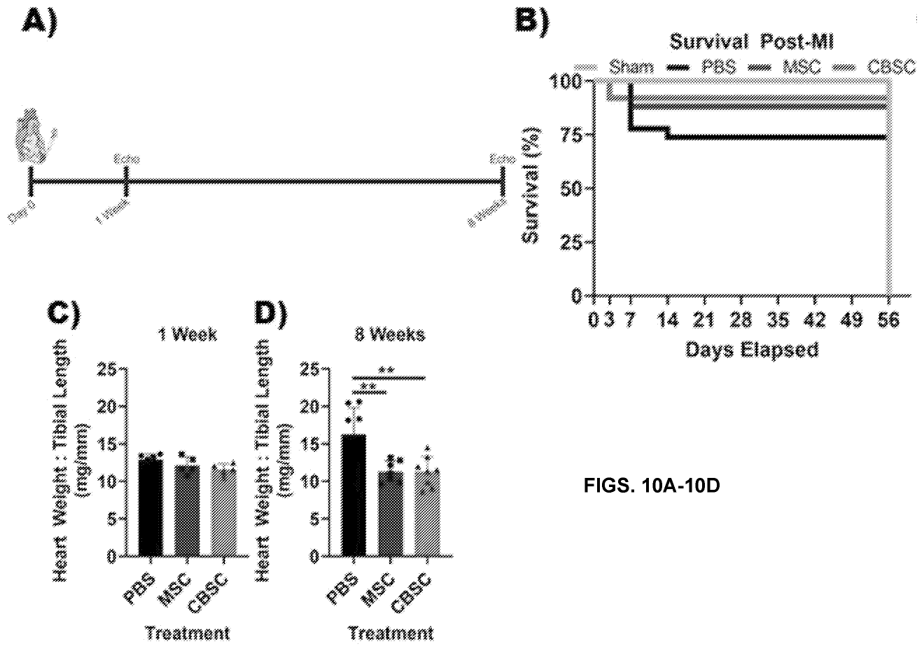

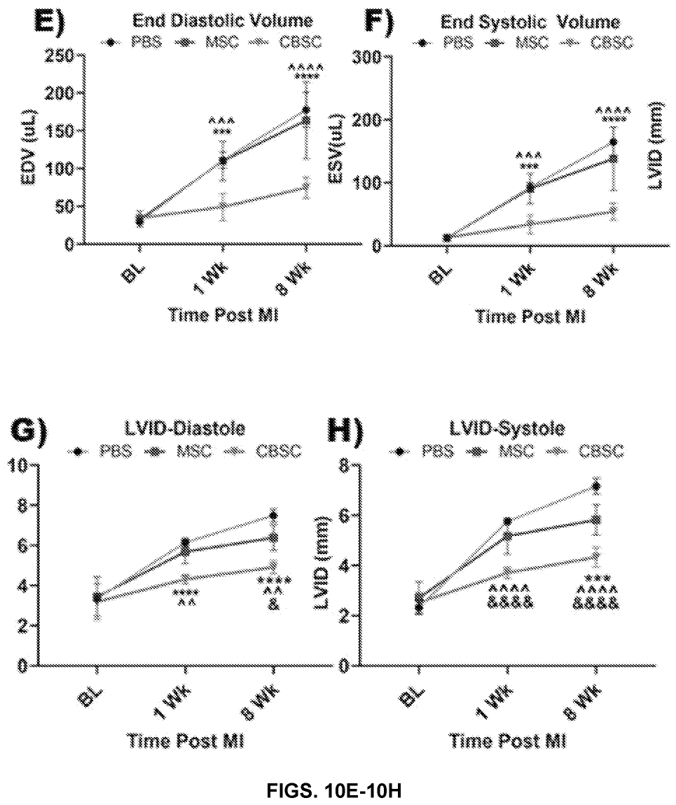

FIGS. 10A-10I are a series of graphs, images and a schematic representation demonstrating that CBSC cell therapy blunts adverse cardiac remodeling during acute and chronic injury states.

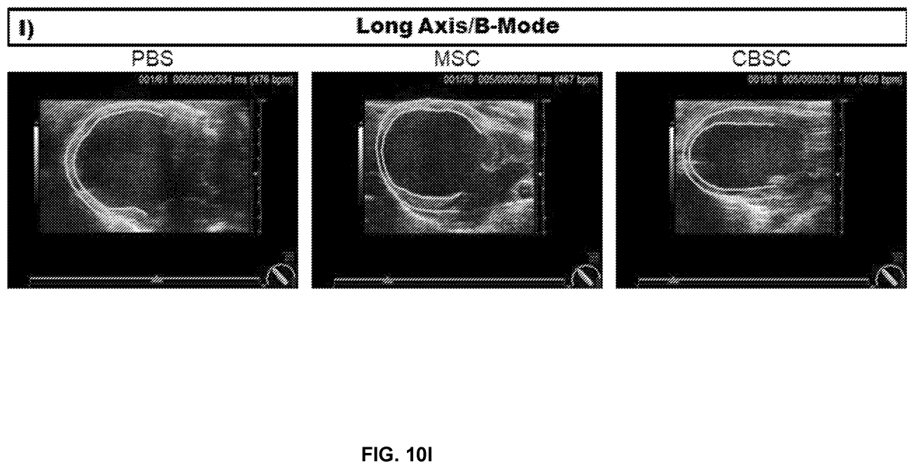

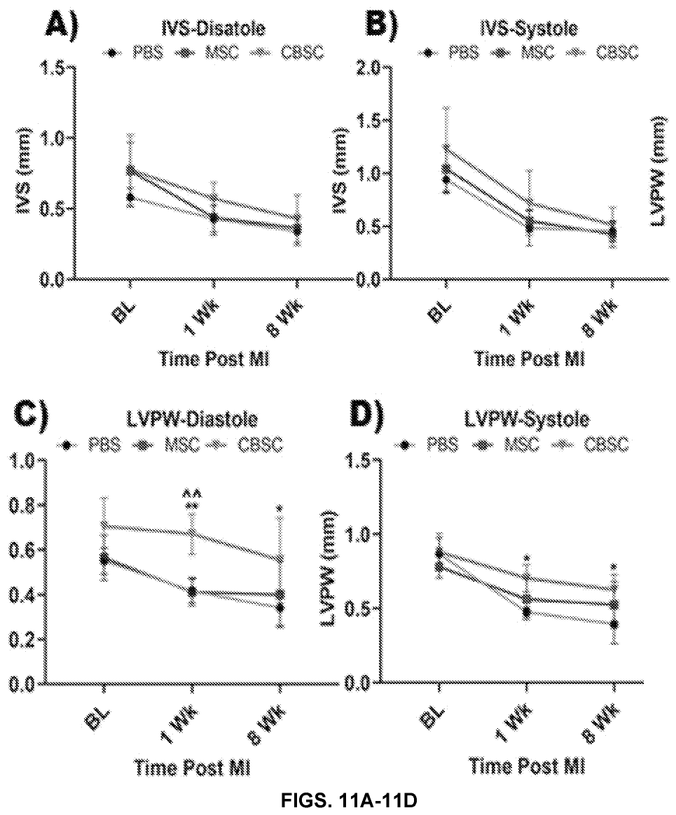

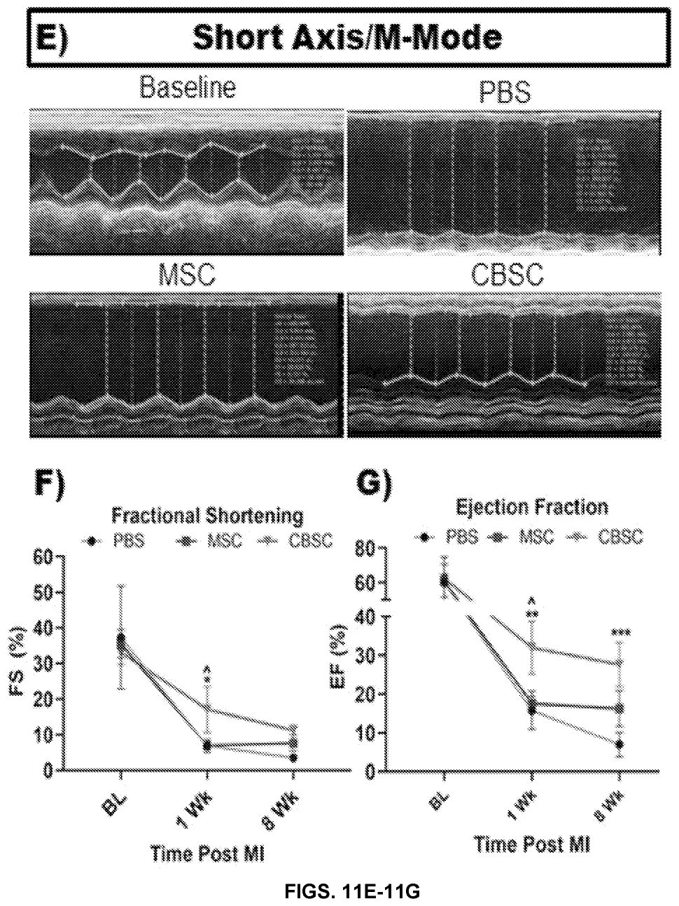

FIGS. 11A-11G are a series of graphs and images demonstrating that CBSCs' Preserve myocardial wall thickness resulting in improved cardiac function during acute and chronic injury states.

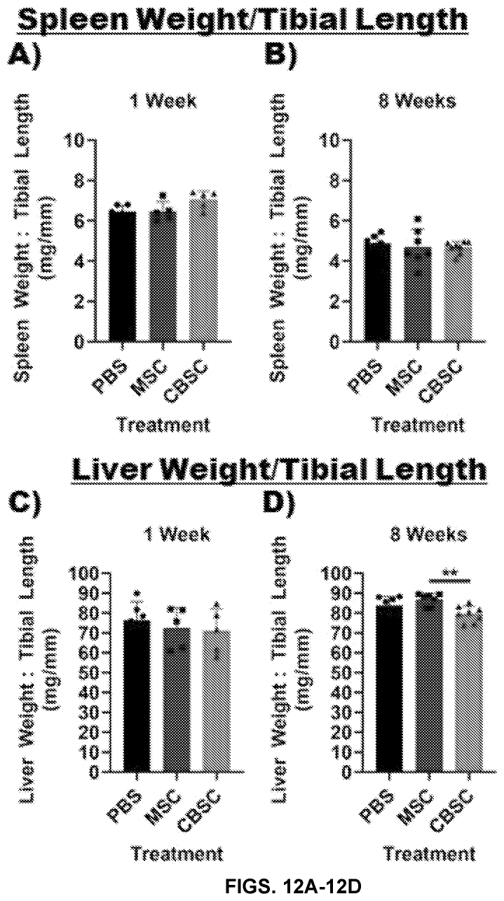

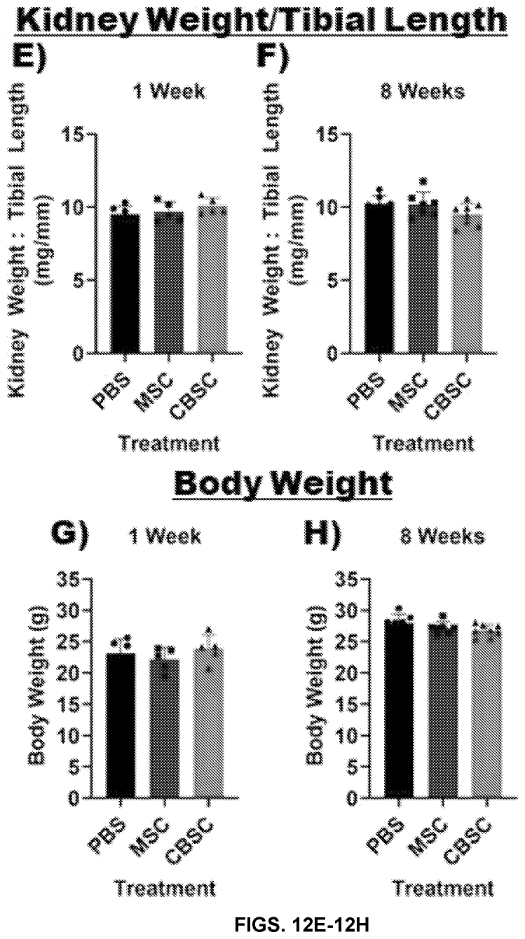

FIGS. 12A-12J are a series of graphs demonstrating that Systemic organ morphology is not altered by cell therapy 1—or 8-week post-MI.

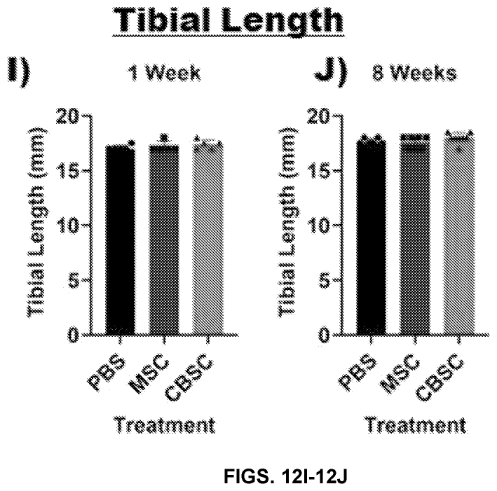

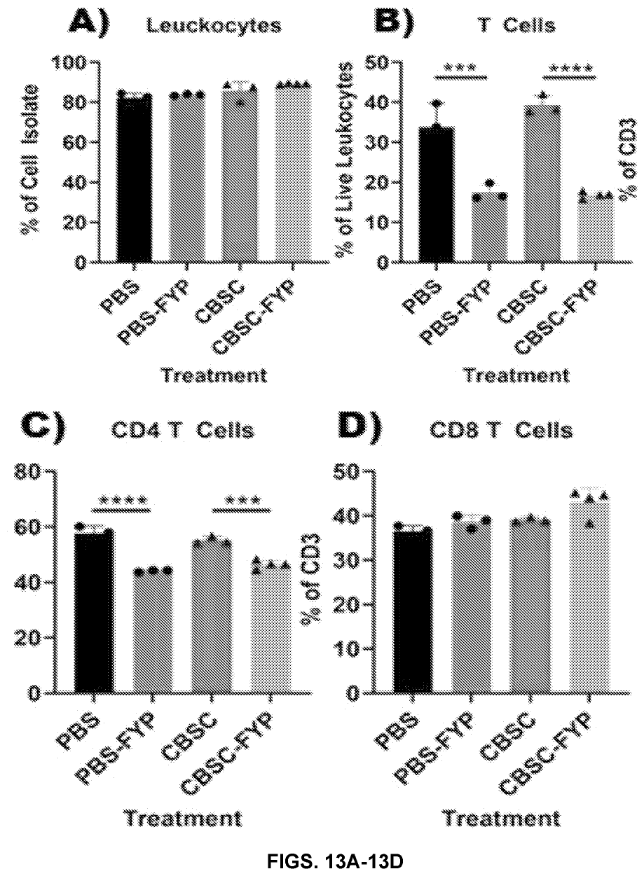

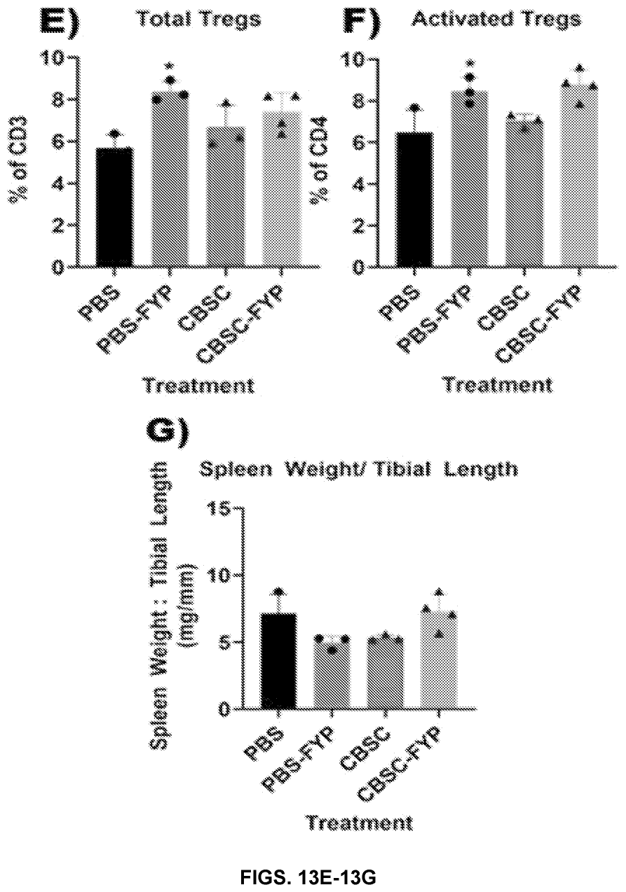

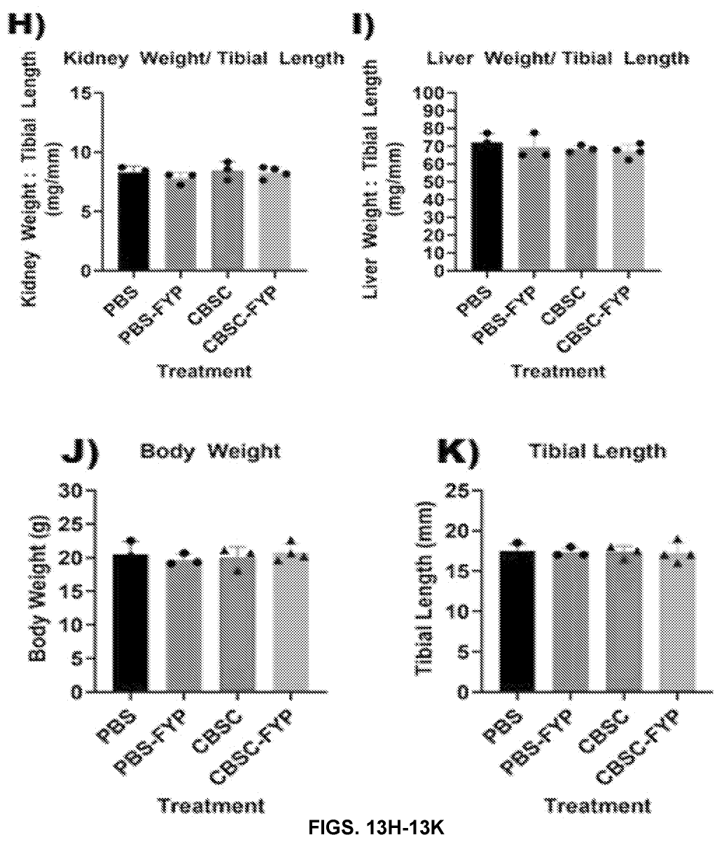

FIGS. 13A-13K are a series of graphs demonstrating that S1P1 introduction results in Treg expansion in splenic tissue without effecting systemic organ morphology.

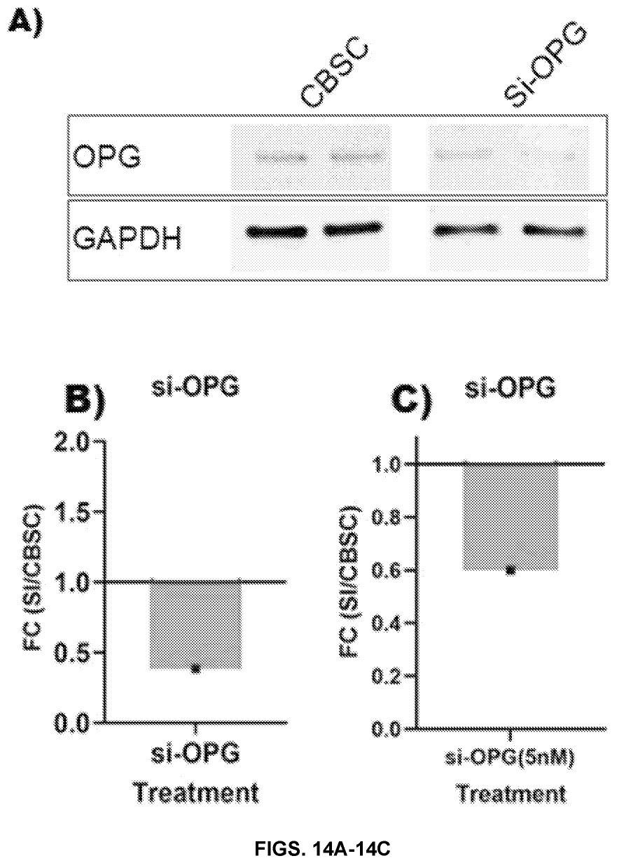

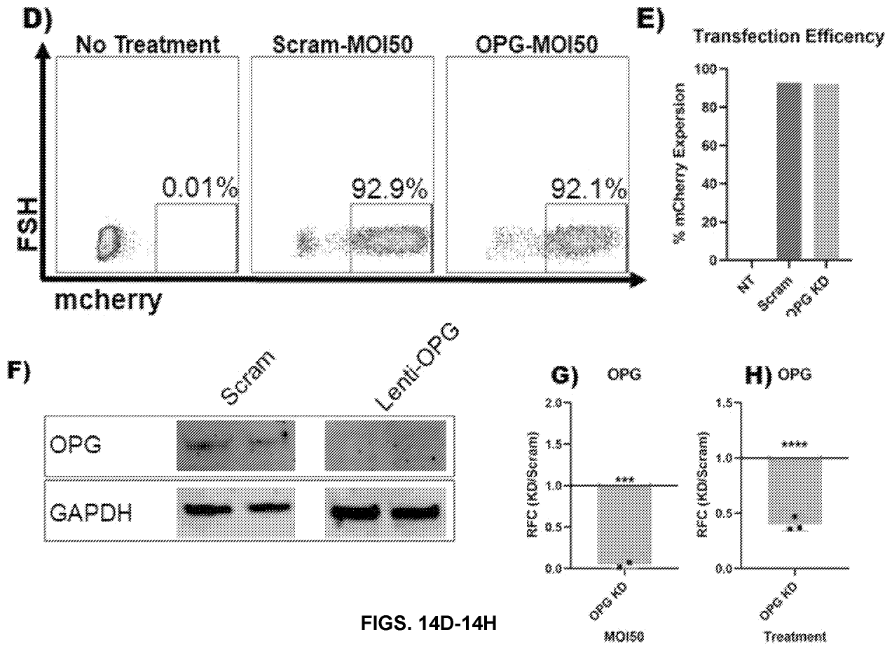

FIGS. 14A-14H are a series of blots and graphs demonstrating the knockdown of osteoprotegrin in CBSCs via siRNA and lentiviral targeted expression.

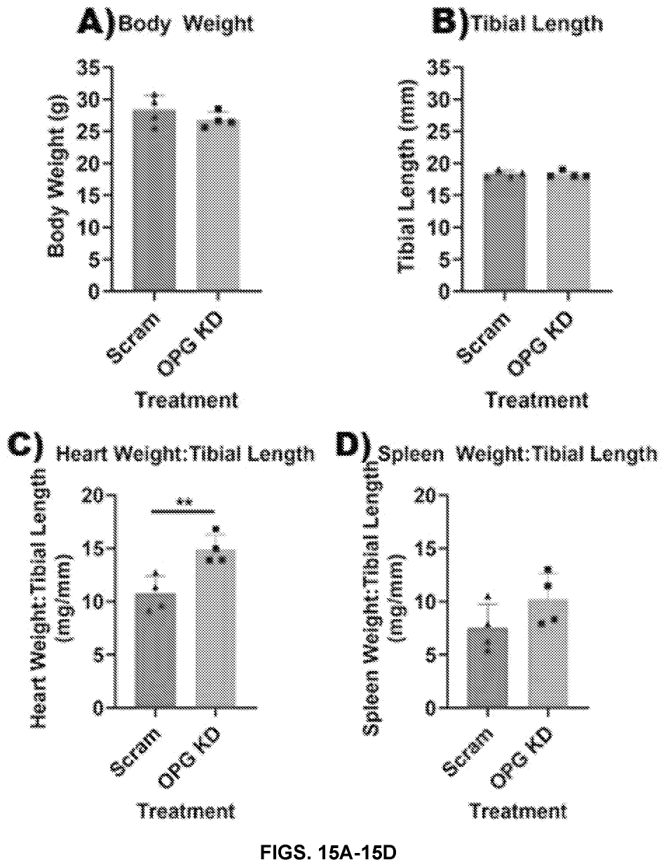

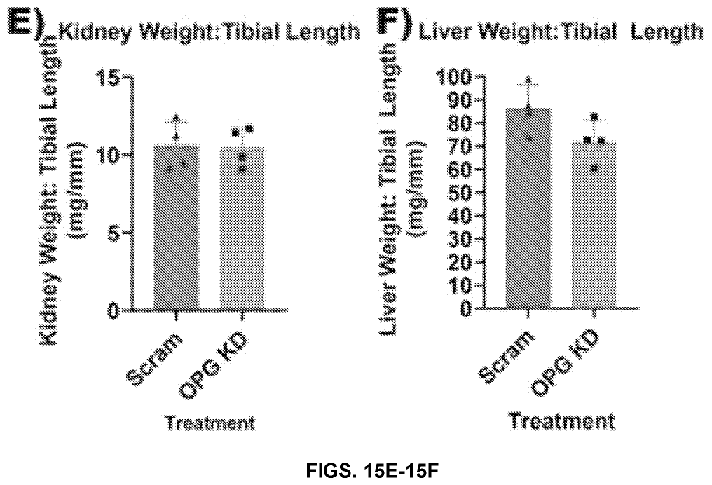

FIGS. 15A-15F are a series of graphs demonstrating that CBSC depletion of OPG solicits cardiac hypertrophy without affecting systemic organ morphology.

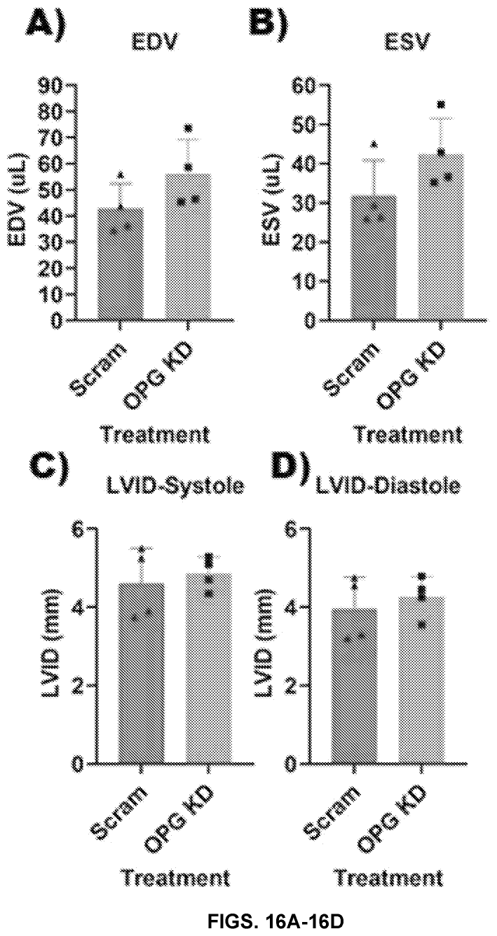

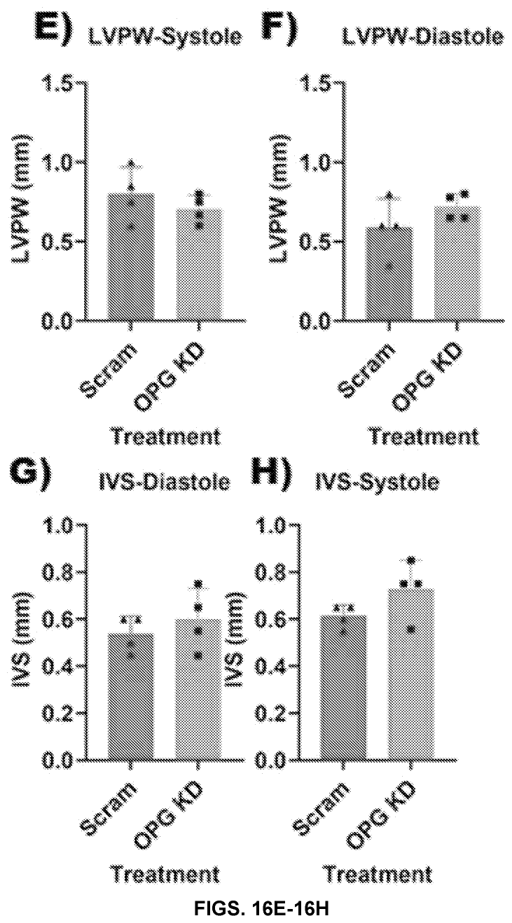

FIGS. 16A-16J are a series of graphs demonstrating that CBSCs deficient in OPG reduce cardiogenic properties of CBSCs in the ischemic heart 1-week post-MI.

### DETAILED DESCRIPTION

The exact role T-lymphocytes serve in modulating cardiac repair is variable and highly dependent upon the T-cell subset population of investigation. In general, CD3+ T-cell populations can be further characterized into CD4⁺ T helper cell populations or CD8⁺ cytotoxic T-cells, both of which have been shown to negatively contribute to cardiac wound healing processes.⁹⁻¹¹ However, a smaller subset of CD4⁺ T-cells retaining FoxP3 transcription factor expression are referred to as T-regulatory Cells (Tregs).¹² Tregs are traditionally described as the main immunosuppressive cell type in the peripheral immune system and are responsible for dampening other T cell subset responses.¹³⁻¹⁵ However, recent reports identify Treg cells possess extra-immunological processes that can maintain tissue homeostasis, earning the designation of Tissue Tregs.¹⁶ Recently, a cardiac specific Treg population that responds to ischemic injury (MI and IR) has been identified.¹⁷ Cardiac Treg populations are phenotypically distinct from other Treg populations that occupy the skeletal muscle, skin, and peripheral lymphoid organs. The ablation of these populations or the blockage of their recruitment to the MI heart compromises cardiac wound healing processes, identifying the necessity these populations have in orchestrating myocardial repair post-MI.¹⁷ In contradiction, a previous report identified that Tregs undergo phenotypic, malfunctional conversions during chronic ischemic injury, resulting in pathogenic, TNFRI⁺ Treg establishment that results in adverse cardiac remodeling and function.¹⁸ Identifying a therapeutic that can modulate the expansion and sustainment of pro-reparative, TNFRII⁺ Tregs in the ischemic heart can greatly augment cardiac wound healing processes.

Recently, the inventors have shown in an MI mouse model that Cortical Bone Derived Stem Cells (CBSCs) are a phenotypically and functional distinct from cardiac-derived stem cells (CDCs) and mesenchymal stem cells (MSCs). CBSCs have superior engraftment (14 days post-M), survival, proliferative, differentiation, and immunomodulatory capabilities compared to CDCs and MSCs.¹⁹ Additionally, intramyocardial injection of CBSCs expands the residence of pro-reparative immune cell subsets, specifically M2 Macrophages (MO) and CD4⁺ T-helper cell populations in the infarcted heart 1-week post-MI in addition to mediating fibroblast maturation following injury. These reports identify that CBSC cell therapy is an ideal cell type to immune mediated cardiogenic, wound healing process in the post-MI heart.^{4,6,20,21}

In this study, the effects of intramyocardial injection of CBSC cell therapy on T-cell population dynamics during acute (1 week) and chronic (8 weeks) stages of ischemic injury in the MI heart were investigated. Additionally, the direct effects paracrine secretome has on T-cell populations dynamics were identified, with an increased emphasis on how CBSC secretome can mediate the expansion, proliferation, and phenotype of Treg cell populations. The results showed that CBSC secretome is enriched in Tnfrsf11b (OPG), a decoy receptor of TNFR mediated signaling,²²⁻²⁴ when compared to MSCs—an ideal comparison given the proximity of these bone marrow derived stem cells have to the hard bone that is the source of CBSCs and the previously identified immune privilege properties previously identified in MSCs.¹⁹ Ablating Treg localization to the heart or CBSC produced OPG greatly compromises Treg cell expansion, recruitment, and presence of pro-reparative Treg signatures occupying the infarcted heart, in turn compromising cardiac wound healing and function.

Accordingly, cortical bone derived stem cells (CBSCs) cell therapy is administered to subjects in need thereof, to mediate myocardial homeostasis and cardiac wound healing process post-MI via the direct modulation of Treg population dynamics and function. In certain embodiments, a composition comprises a therapeutically effective amount of cortical bone derived stem cells (CBSCs) and/or CBSC secretome.

### Methods of Treatment

In certain embodiments, the methods described herein involve intramyocardial transplantation of CBSCs. Such therapeutic methods may repair and regenerate damaged myocardium and restore cardiac function after, for example, acute myocardial infarction and/or other ischemic or reperfusion related injuries. Methods generally include contacting cardiac tissues with a composition comprising CBSCs, CBSC secretome compositions or the combination thereof. Contacting may occur via injection methods known in the art and described herein.

Accordingly, in certain embodiments, a method of promoting myocardial homeostasis and cardiac wound healing in a subject in need thereof, comprises administering to the subject a therapeutically effective amount of cortical bone derived stem cells (CBSCs); thereby, promoting myocardial homeostasis and cardiac wound healing in a subject. In certain embodiments, the CBSCs are administered via intramyocardial injection. In certain embodiments, the CBSCs are autologous, allogeneic, haplotype matched, haplotype mismatched, haplo-identical, xenogeneic or combinations thereof. In certain embodiments, the method further comprises administering a therapeutically effective amount of a CBSC secretome. In certain embodiments, the CBSC secretome comprises osteoprotegrin (OPG) and/or insulin-like growth factor-binding protein 5 (IGFBP-5). In certain embodiments, the CBSCs and/or secretome induce T regulatory (Treg) cells. In certain embodiments, the CBSCs and/or secretome mediate recruitment and expansion of Treg from peripheral T cell stores into the subject's infarcted heart. In certain embodiments, the CBSCs and/or secretome modulate the inflammatory microenvironment of the subject's infarcted heart. In certain embodiments, the induced Treg cells are TNFRII⁺. In certain embodiments, the CBSCs are expanded ex vivo. In certain embodiments, the CBSC secretome is enriched ex vivo by culturing the CBSCs for at least 12 hours. In certain embodiments, the method further comprises culturing Treg cells ex vivo with the CBSC secretome. In certain embodiments, the Treg cells are TNFRII⁺. In certain embodiments, the TNFRII⁺ Treg cells are adoptively transferred to the subject.

In certain embodiments, a method for restoring cardiac function comprises introducing an effective amount of a composition comprising CBSCs, CBSC secretome compositions or the combination thereof, and a pharmaceutically acceptable carrier into a heart of a subject in need thereof. Restoration of cardiac function may include partial or complete restoration. In one embodiment, at least 50% of cardiac function is restored compared to a patient who does not receive such treatment. In another embodiment, about 50%, 60%, 70%, 75%, 80%, 85%, 90%, 95%, or 100% of cardiac function is restored. A subject receiving treatment may also be tested in various ways for cardiac health and have an improved result observed by echocardiography, multi-gated acquisition scan (MUGA) scan, nuclear stress test, radionuclide angiography, left ventricular angiography, MRI or ECG. In one embodiment, a patient's cardiac function does not worsen.

In certain embodiments, a method of inducing cardiomyocyte regeneration, cardiac repair, vasculogenesis or cardiomyocyte differentiation, comprises contacting a composition comprising CBSCs, CBSC secretome compositions or the combination thereof with injured heart tissue. In one embodiment, the CBSCs represent at least about 75%, at least about 80%, at least about 85%, at least about 90%, at least about 95%, at least about 98%, at least about 99%, or 100% of the cells in the composition.

In such methods, a subject may be diagnosed with, or at risk for, myocardial infarction, chronic coronary ischemia, arteriosclerosis, congestive heart failure, dilated cardiomyopathy, restenosis, coronary artery disease, heart failure, arrhythmia, angina, atherosclerosis, hypertension, or myocardial hypertrophy. In one embodiment, the subject is diagnosed with myocardial infarction. In another embodiment, the subject has, or is at risk for, heart failure.

Where compositions such as those described herein are utilized for treatment of a subject, introducing or contacting the composition with the heart of the subject can occur by implanting the composition into cardiac tissue of the subject. Alternatively, introducing or contacting the composition can occur via injecting the composition into the subject using conventional techniques in the art. Cardiac tissue to be treated according to the present methods includes, for example, myocardium, endocardium, epicardium, connective tissue in the heart, and nervous tissue in the heart. Animals such as mammals represent subjects to be treated with the presently disclosed compositions and methods. In one embodiment, the subject is a human, a veterinary animal, a primate, a domesticated animal, a reptile, or an avian. For example, a human subject may be treated with the disclosed compositions to restore cardiac function and to treat one or more heart-related conditions.

In certain embodiments, a method of preventing or treating a patient suffering from myocardial infarction, chronic coronary ischemia, arteriosclerosis, congestive heart failure, dilated cardiomyopathy, restenosis, coronary artery disease, heart failure, arrhythmia, angina, atherosclerosis, hypertension, or myocardial hypertrophy comprises administering a composition comprising CBSCs, CBSC secretome compositions or the combination thereof, and a pharmaceutically acceptable carrier.

Generally, a subject upon which the methods of the invention are to be performed will have been diagnosed with myocardial infarction, chronic coronary ischemia, arteriosclerosis, congestive heart failure, dilated cardiomyopathy, restenosis, coronary artery disease, heart failure, arrhythmia, angina, atherosclerosis, hypertension, or myocardial hypertrophy. Alternatively, it will have been determined that a subject upon which the methods of the invention are performed is at risk for myocardial infarction, chronic coronary ischemia, arteriosclerosis, congestive heart failure, dilated cardiomyopathy, restenosis, coronary artery disease, heart failure, arrhythmia, angina, atherosclerosis, hypertension, or myocardial hypertrophy based on assessment of the heart tissue and/or family history. In one embodiment, a subject has been diagnosed with myocardial infarction or at risk for heart failure.

One in this field of endeavor would understand that methods of prevention are intended for subjects that have a family history of heart attacks or may physically be predisposed to heart attacks. Thus, prevention encompasses administration of compositions embodied herein to a subject to prevent damage to the subject's heart and/or to prevent acute myocardial infarction. A subject that has been treated with such methods may experience an overall improvement in health. Additionally, cardiac function may be restored and/or improved as described above compared to lack of treatment.

In accordance with one embodiment, a composition comprising CBSCs, CBSC secretome compositions or the combination thereof, is introduced into the cardiac tissue or cells a subject. Briefly, this method may be performed as follows. CBSCs are isolated as described in US 2016/0152952 incorporated herein by reference in its entirety. Once isolated, the CBSCs can be purified and/or expanded. The isolated cells can then be formulated as a composition (medicament) comprising the CBSCs, CBSC secretome compositions or the combination thereof, along with, for example, a pharmaceutically acceptable carrier. The composition (medicament) so formed can then be introduced into the heart tissue of a subject.

A subject to be treated with the disclosed compositions and methods will have been diagnosed as having, or being at risk for, a heart condition, disease, or disorder. Introduction of the composition can be according to methods described herein or known in the art. For example, the CBSCs, CBSC secretome compositions or the combination thereof, can be administered to a subject's heart by way of direct injection delivery or catheter delivery. Introduction of CBSCs, CBSC secretome compositions can be a single occurrence or can occur more than one time over a period of time selected by the attending physician.

The time course and number of occurrences of CBSC implantation into a subject's heart can be dictated by monitoring generation and/or regeneration of cardiac tissue, where such methods of assessment and devisement of treatment course is within the skill of the art of an attending physician.

Cardiac tissue into which CBSCs, CBSC secretome compositions can be introduced includes, but is not limited to, the myocardium of the heart (including cardiac muscle fibers, connective tissue (endomysium), nerve fibers, capillaries, and lymphatics); the endocardium of the heart (including endothelium, connective tissue, and fat cells); the epicardium of the heart (including fibroelastic connective tissue, blood vessels, lymphatics, nerve fibers, fat tissue, and a mesothelial membrane consisting of squamous epithelial cells); and any additional connective tissue (including the pericardium), blood vessels, lymphatics, fat cells, progenitor cells (e.g., side-population progenitor cells), and nervous tissue found in the heart. Cardiac muscle fibers are composed of chains of contiguous heart-muscle cells (cardiomyocytes), joined end to end at intercalated disks. These disks possess two kinds of cell junctions: expanded desmosomes extending along their transverse portions, and gap junctions, the largest of which lie along their longitudinal portions. Each of the above tissues can be selected as a target site for introduction of CBSCs, CBSC secretome compositions, either individually or in combination with other tissues.

A determination of the need for treatment will typically be assessed by a history and physical exam consistent with the myocardial defect, disorder, or injury at issue. Subjects with an identified need of therapy include those with diagnosed damaged or degenerated heart tissue (i.e., heart tissue which exhibits a pathological condition) or which are predisposed to damaged or degenerative heart tissue. Causes of heart tissue damage and/or degeneration include, but are not limited to, chronic heart damage, chronic heart failure, damage resulting from injury or trauma, damage resulting from a cardiotoxin, damage from radiation or oxidative free radicals, damage resulting from decreased blood flow, and myocardial infarction (such as a heart attack). In one embodiment, a subject in need of treatment according to the methods described herein has been diagnosed with degenerated heart tissue resulting from a myocardial infarction or heart failure.

It should be recognized that methods disclosed herein can be practiced in conjunction with existing myocardial therapies to effectively treat or prevent disease. The methods, compositions, and devices of the invention can include concurrent or sequential treatment with non-biologic and/or biologic drugs.

The subject receiving cardiac implantation of CBSCs according to the methods described herein will usually have been diagnosed as having, or being at risk for, a heart condition, disease, or disorder. The methods of treatment can be useful to alleviate the symptoms of a variety of disorders, such as disorders associated with aberrant cell/tissue damage, ischemic disorders, and reperfusion related disorders. For example, the methods are useful in alleviating a symptom of myocardial infarction, chronic coronary ischemia, arteriosclerosis, congestive heart failure, dilated cardiomyopathy, restenosis, coronary artery disease, heart failure, arrhythmia, angina, atherosclerosis, hypertension, myocardial hypertrophy, or a combination thereof. The methods of the invention can also be useful to prevent the symptoms of a variety of disorders, such as disorders associated with aberrant cell/tissue damage, ischemic disorders, and reperfusion related disorders. For example, the methods are useful in preventing a symptom of myocardial infarction, chronic coronary ischemia, arteriosclerosis, congestive heart failure, dilated cardiomyopathy, restenosis, coronary artery disease, heart failure, arrhythmia, angina, atherosclerosis, hypertension, myocardial hypertrophy, or a combination thereof. The condition, disease, or disorder can be diagnosed and/or monitored, typically by a physician using standard methodologies.

Alleviation of one or more symptoms of the cardiovascular condition, disease, or disorder indicates that the composition confers a clinical benefit, such as a reduction in one or more of the following symptoms: shortness of breath, fluid retention, headaches, dizzy spells, chest pain, left shoulder or arm pain, and ventricular dysfunction. One would understand that a reduction of one more of the symptoms need not be 100% to provide therapeutic benefit to the subject being treated. Thus, in one embodiment, a reduction of about 50%, about 60%, about 70%, about 80%, about 90%, or more of one or more such symptoms may provide sufficient therapeutic relief to a patient.

With respect to methods of prevention, one of skill in the art would understand that prevention does not necessarily mean that a patient never experiences cardiac damage. Rather, prevention includes, but is not limited to, delay of onset of one or more symptoms compared to a lack of treatment. In one non-limiting example, a patient who has a family history of fatal heart attacks by 50 years of age may experience one or more symptoms described herein, but not experience a fatal heart attack or may experience a less severe heart attack compared to lack of treatment.

Cardiac cell/tissue damage is characterized, in part, by a loss of one or more cellular functions characteristic of the cardiac cell type which can lead to eventual cell death. For example, cell damage to a cardiomyocyte results in the loss of contractile function of the cell resulting in a loss of ventricular function of the heart tissue. An ischemic or reperfusion related injury results in tissue necrosis and scar formation. Injured myocardial tissue is defined for example by necrosis, scarring, or yellow softening of the myocardial tissue. Injured myocardial tissue leads to one or more of several mechanical complications of the heart, such as ventricular dysfunction, decreased forward cardiac output, as well as inflammation of the lining around the heart (i.e., pericarditis). Accordingly, regenerating injured myocardial tissue according to the methods described herein can result in histological and functional restoration of the tissue.

In embodiments, the methods described herein promote generation and/or regeneration of heart tissue, and/or promote endogenous myocardial regeneration of heart tissue in a subject. Promoting generation of heart tissue generally includes, but is not limited to, activating, enhancing, facilitating, increasing, inducing, initiating, or stimulating the growth and/or proliferation of heart tissue, as well as activating, enhancing, facilitating, increasing, inducing, initiating, or stimulating the differentiation, growth, and/or proliferation of heart tissue cells. Thus, the methods include, for example, initiation of heart tissue generation, as well as facilitation or enhancement of heart tissue generation already in progress. Differentiation is generally understood as the cellular process by which cells become structurally and functionally specialized during development. Proliferation and growth, as used herein, generally refer to an increase in mass, volume, and/or thickness of heart tissue, as well as an increase in diameter, mass, or number of heart tissue cells. The term generation is understood to include the generation of new heart tissue and the regeneration of heart tissue where heart tissue previously existed.

Generation of new heart tissue and regeneration of heart tissue, resultant from the therapeutic methods embodied herein, can be detected and/or measured using conventional procedures in the art. Such procedures include, but are not limited to, Western blotting for heart-specific proteins, electron microscopy in conjunction with morphometry, simple assays to measure rate of cell proliferation (including trypan blue staining, the Cell Titer-Blue cell viability assay from Promega (Madison, Wis.), the MTT cell proliferation assay from American Type Culture Collection (ATCC), differential staining with fluorescein diacetate and ethidium bromide/propidium iodide, estimation of ATP levels, flow-cytometry assays, etc.), and any of the methods, molecular procedures, and assays disclosed herein.

CBSCs can be isolated from bone, purified, and cultured as described as described in US 2016/0152952 incorporated herein by reference in its entirety. Additional art-recognized methods of isolating, culturing, and differentiating stems cells are generally known in the art (see, e.g., Lanza et al., eds. (2004) Handbook of Stem Cells, Academic Press, ISBN 0124366430; Lanza et al., eds. (2005) Essentials of Stem Cell Biology, Academic Press, ISBN 0120884429; Saltzman (2004) Tissue Engineering: Engineering Principles for the Design of Replacement Organs and Tissues, Oxford ISBN 019514130X; Vunjak-Novakovic and Freshney, eds. (2006) Culture of Cells for Tissue Engineering, Wiley-Liss, ISBN 0471629359; Minuth et al. (2005) Tissue Engineering: From Cell Biology to Artificial Organs, John Wiley & Sons, ISBN 3527311866). Such methods can be utilized directly or adapted for use with the CBSCs described herein.

It will be appreciated that the time between isolation, culture, expansion, and/or implantation may vary according to a particular application and/or a particular subject. Incubation (and subsequent replication and/or differentiation) of a composition containing CBSCs can be, for example, at least in part in vitro, substantially in vitro, at least in part in vivo, or substantially in vivo. Determination of optimal culture time may be empirically determined.

CBSCs can be derived from bone of the same or different species as the transplant recipient. For example, CBSCs can be derived from an animal, including but not limited to, mammals, reptiles, and avians such as, for example, horses, cows, dogs, cats, sheep, pigs, chickens, and humans. It is also contemplated that autologous CBSCs may be obtained from the subject, into which the CBSCs are re-introduced. Such autologous CBSCs may be expanded and/or transformed before re-introduction to the host.

CBSCs may be selected and prepared for transplantation. In one aspect, therapeutic CBSCs are expanded ex vivo (or in vitro) using, for example, methods used to culture CBSCs as described in the examples section which follows. Alternatively, these cells can be expanded in vivo (i.e., after implantation). These cells can also be used for future transplantation procedures. The screened and isolated cells may, optionally, be further enriched for CBSCs prior to transplantation. Methods to select for CBSC, are well known in the art (e.g., MoFlow Cell Sorter). Methods as described herein and in US 2016/0152952, incorporated herein by reference in its entirety, are used. Alternatively, a sample of the CBSC rich culture can be implanted without further enrichment.

Isolated CBSCs can optionally be transformed with a heterologous nucleic acid so as to express a bioactive molecule or heterologous protein or to overexpress an endogenous protein. In certain embodiments, the CBSCs comprise and expression vector encoding CBSCs comprising an expression vector encoding osteoprotegrin (OPG) and/or insulin-like growth factor-binding protein 5 (IGFBP-5). In certain embodiments, the isolated nucleic acid sequences osteoprotegrin (OPG) and/or insulin-like growth factor-binding protein 5 (IGFBP-5) are included in at least one expression vector selected from the group consisting of: a lentiviral vector, an adenovirus vector, an adeno-associated virus vector, a vesicular stomatitis virus (VSV) vector, a pox virus vector, and a retroviral vector. In certain embodiments, the expression vector comprises: a lentiviral vector, an adenoviral vector, or an adeno-associated virus vector. In certain embodiments, the adeno-associated virus (AAV) vector is AV1, AAV2, AAV3, AAV4, AAV5, AAV6, AAV7, AAV8, AAV9, AAV10, AAV11, AAVDJ, or AAVDJ/8. In certain embodiments, the vector comprising the nucleic acid further comprises a promoter. In certain embodiments, the promoter comprises a ubiquitous promoter, a tissue-specific promoter, an inducible promoter or a constitutive promoter.

In the context of an expression vector, the vector can be readily introduced into a host cell, e.g., mammalian, bacterial, yeast or insect cell by any method in the art. Coding sequences for a desired peptide of the disclosure may be codon optimized based on the codon usage of the intended host cell in order to improve expression efficiency as demonstrated herein. Codon usage patterns can be found in the literature (Nakamura et al., 2000, Nuc Acids Res. 28:292). Representative examples of appropriate hosts include bacterial cells, such as streptococci, staphylococci, E. coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, HEK 293 and Bowes melanoma cells; and plant cells.

Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

The expression vector can be transferred into a host cell by physical, biological or chemical means. Transformation of stem cells, including CBSCs, can be conducted using conventional methods in the art. In one non-limiting example, CBSCs may be genetically modified to expresses a fluorescent protein marker (e.g., GFP, eGFP, BFP, CFP, YFP, RFP, etc.). Marker protein expression can be especially useful in implantation scenarios, as described herein, so as to monitor CBSC placement, retention, and replication in target tissue. As another example, CBSCs may be transfected with one or more genetic sequences that are capable of reducing or eliminating an immune response in the host.

In certain embodiments, a method for enhancing cardiac function in a subject in need thereof, comprises introducing a composition comprising CBSCs, CBSC secretome compositions or the combination thereof, into the heart of a subject. In certain embodiments, the CBSC secretome compositions are administered directly into the heart or systemically. In certain embodiments, CBSCs, CBSC secretome compositions or the combination thereof, are directly introduced into, or contacted with, cardiac tissue and/or cells. Introduction to the tissues or cells of a subject may occur ex vivo or in vivo. In one embodiment, compositions containing isolated cells are directly implanted into cardiac tissue of the subject, in vivo.

Improving or enhancing cardiac function generally refers to improving, enhancing, augmenting, facilitating or increasing the performance, operation, or function of the heart and/or circulatory system of a subject. Improving or enhancing cardiac function may also refer to an improvement in one or more of the following symptoms: chest pain (typically radiating to the left arm or left side of the neck), shortness of breath, nausea, vomiting, palpitations, sweating, and anxiety. The number of cells introduced into the heart tissue of the subject can be that amount sufficient to forming endothelial cells, smooth muscle cells and/cardiomyocytes. An improvement in cardiac function may be readily assessed and determined based on known procedures including, but limited to, an electrocardiogram (ECG), echocardiography, measuring volumetric ejection fraction using magnetic resonance imaging (MRI) and/or one or more blood tests. The most often used markers for blood tests are the creatine kinase-MB (CK-MB) fraction and the troponin levels.

Introduction of cell-containing compositions can occur as a single event or over a time course of treatment. For example, compositions can be administered daily, weekly, bi-weekly, or monthly. For treatment of acute conditions, the time course of treatment generally will be at least several days. Certain conditions may extend treatment from several days to several weeks. For example, treatment could extend over one week, two weeks, or three weeks. For chronic conditions or preventative treatments, treatment regimens may extend from several weeks to several months or even a year or more.

### Administration of CBSCs

The CBSCs can be administered by any appropriate route. In various embodiments, the CBSCs are systemically administered, e.g., intravenously, intra-arterially, or administered directly to the tissue of interest for treatment or repair.

In some embodiments, the CBSCs are administered locally, e.g., directly to cardiac tissue. As appropriate, the CBSCs can be engrafted or transplanted into and/or around the tissue of interest, e.g., cardiac tissues. When engrafted or transplanted into and/or in the vicinity of one or more tissues of interest (e.g., cardiac tissues), the CBSCs are administered within or within sufficient proximity of inflamed or damaged lesions in tissue to mitigate and/or reverse of damage and/or destruction of the tissue. For example, the CBSCs are engrafted or transplanted into or within sufficient proximity to the tissue of interest to prevent, reduce or inhibit damage and/or destruction to the tissues.

As appropriate, injections of CBSCs can be done after local anesthetics (e.g., lidocaine, bupivacaine) have been administered. It is also possible to inject the CBSCs in conjunction with local anesthetics added to the cell suspension. Injections can also be made with the subject under general anesthesia with or without the use of local anesthetic agents (e.g., lidocaine). In one aspect, the cells may be transplanted along with a carrier material, such as collagen or fibrin glue or other scaffold materials. Such materials may improve cell retention and integration after implantation. Exemplary materials and methods for employing them are known in the art and are contemplated herein (see, e.g., Saltzman (2004) Tissue Engineering: Engineering Principles for the Design of Replacement Organs and Tissues, Oxford ISBN 019514130X; Vunjak-Novakovic and Freshney, eds. (2006) Culture of Cells for Tissue Engineering, Wiley-Liss, ISBN 0471629359; and Minuth et al. (2005) Tissue Engineering: From Cell Biology to Artificial Organs, John Wiley & Sons, ISBN 3527311866).

In certain embodiments, engraftment or transplantation of the CBSCs can be facilitated using a matrix or caged depot. For example, the CBSCs can be engrafted or transplanted in a “caged cell” delivery device wherein the cells are integrated into a biocompatible and/or biologically inert matrix (e.g. a hydrogel or other polymer or any device) that restricts cell movement while allowing the cells to remain viable. Synthetic extracellular matrix and other biocompatible vehicles for delivery, retention, growth, and differentiation of CBSCs are known in the art and find use in the present methods. See, e.g., Prestwich, J Control Release. 2011 Apr. 14, PMID 21513749; Perale, et al., Int J Artif Organs. (2011) 34(3):295-303; Suri, et al., Tissue Eng Part A. (2010) 16(5): 1703-16; Khetan, et al., J Vis Exp. (2009) October 26; (32). pii: 1590; Salinas, et al., J Dent Res. (2009) 88(8):681-92; Schmidt, et al., J Biomed Mater Res A. (2008) 87(4):1113-22 and Xin, et al., Biomaterials (2007) 28:316-325.

As appropriate or desired, the engrafted or transplanted CBSCs can be modified to facilitate retention of the CBSCs at the region of interest or the region of delivery. In other embodiments, the region of interest for engraftment or transplantation of the cells is modified in order to facilitate retention of the CBSCs at the region of interest or the region of delivery, e.g. cytokines, growth factors etc. Therapeutic cells may be implanted into a subject using conventional methods (see, for example, Orlic et al. (2001) Nature, 410(6829): 701-705). For example, cells, or compositions comprising cells, may be introduced via direct injection (e.g., intermyocardial or intercoronary injection) or catheter-based delivery (e.g., intermyocardial, intercoronary, or coronary sinus delivery). Intercoronary catheter delivery directly injects cells into heart tissue.

The number of cells introduced into the heart tissue of the subject can be that amount sufficient to improve cardiac function, increase cardiomyocyte formation, and/or increase mitotic index of cardiomyocytes. For example, an effective amount may increase cardiomyocyte formation, increase cardiomyocyte proliferation, increase cardiomyocyte cell cycle activation, increased mitotic index of cardiomyocytes, increase myofilament density, increase borderzone wall thickness, or a combination thereof. An effective amount may form endothelial cells, smooth muscle cells, cardiomyocytes, or a combination thereof.

In some embodiments, at least about 1 million CBSCs/kg subject are administered or engrafted, e.g., at least about 2 million, 2.5 million, 3 million, 3.5 million, 4 million, 5 million, 6 million, 7 million, 8 million, 9 million or 10 million CBSCs/kg subject are administered. In varying embodiments, about 1 million to about 10 million CBSCs/kg subject, e.g., about 2 million to about 8 million CBSCs/kg are administered or grafted.

In varying embodiments, at least about 5 million CBSCs are administered or engrafted, e.g., at least about 10 million, 15 million, 20 million, 25 million, 30 million, 35 million, 40 million, 45 million, 50 million, 55 million, 60 million, 65 million, 70 million, 75 million, 80 million, 85 million, 90 million, 95 million or 100 million CBSCs are administered or engrafted. In varying embodiments, about 5 million to about 100 million CBSCs are administered or engrafted, e.g., about 10 million to about 80 million CBSCs are administered or engrafted. In varying embodiments, at least about 50 million CBSCs are administered or engrafted, e.g., at least about 100 million, 150 million, 200 million, 250 million, 300 million, 350 million, 400 million, 450 million, 500 million, 550 million, 600 million, 650 million, 700 million, 750 million, 800 million, 850 million, 900 million, 950 million or 1 billion CBSCs are administered or engrafted.

In certain embodiments, the CBSCs are administered, e.g., intravenously, at a rate of about 1 million to about 10 million cells per minute, e.g., at a rate of about 2 million to about 4 million cells per minute, e.g., at a rate of about 2.5 million to about 3.5 million cells per minute.

Treatment or prevention may involve one or multiple injections. For example, CBSCs may be administered to the subject 1, 2, 3, 4, 5, 6, 7, 8, 9, 10, or more times, as appropriate. Subsequent administrations of CBSCs may be administered systemically or locally. If administered locally, multiple injections of CBSCs may be administered to the same or different locations. Multiple injections of CBSCs can be administered daily, weekly, bi-weekly, monthly, bi-monthly, every 3, 4, 5, or 6 months, or annually, or more or less often, as needed by the subject. The frequency of administration of the CBSCs can change over a course of treatment, e.g., depending on how well the engrafted or transplanted CBSCs establish themselves at the site of administration and the responsiveness of the subject. The CBSCs may be administered multiple times over a regime course of several weeks, several months, several years, or for the remainder of the life of the subject, as needed or appropriate.

In certain embodiments, the CBSCs are administered with a CBSC secretome composition. The CBSC secretome composition can be administered, prior to, in conjunction with and after CBSC administration. The administration of the secretome composition can be extended until the CBSCs have differentiated into the appropriate cardiac cell, e.g. cardiac myocyte.

The total amount of cells that are envisioned for use depend upon the desired effect, patient state, and the like, and may be determined by one skilled within the art. Dosages for any one patient depends upon many factors, including the patient's species, size, body surface area, age, the particular CBSCs to be administered, sex, scheduling and route of administration, general health, and other drugs being administered concurrently.

Also provided herein is a composition comprising a population of CBSCs and a pharmaceutically acceptable carrier for treating myocardial infarction, chronic coronary ischemia, arteriosclerosis, congestive heart failure, dilated cardiomyopathy, restenosis, coronary artery disease, heart failure, arrhythmia, angina, atherosclerosis, hypertension, or myocardial hypertrophy, wherein the CBSCs are isolated from the bone or fragments thereof.

Also provided herein is a composition comprising a population of CBSCs and/or a CBSC secretome composition and a pharmaceutically acceptable carrier for inducing cardiac regeneration, wherein the CBSCs are isolated from the bone or fragments thereof. In another embodiment, the compositions increase cardiomyocyte formation, increase cardiomyocyte proliferation, increase cardiomyocyte cell cycle activation, increase mitotic index of cardiomyocytes, increase myofilament density, increase border zone wall thickness, or a combination thereof, when administered to a subject. In another embodiment, the composition treats myocardial infarction, chronic coronary ischemia, arteriosclerosis, congestive heart failure, dilated cardiomyopathy, restenosis, coronary artery disease, heart failure, arrhythmia, angina, atherosclerosis, hypertension, or myocardial hypertrophy when administered to a subject.

One would understand that one or more additional excipients, carriers, adjuvants, cells, etc., may be added to the compositions described herein.

Clinical efficacy can be monitored using any method known in the art. Measurable parameters to monitor efficacy will depend on the condition being treated. For monitoring the status or improvement of one or more symptoms associated with cardiomyopathy, measurable parameters can include without limitation, auditory inspection (e.g., using a stethoscope), blood pressure, electrocardiogram (EKG), magnetic resonance imaging (MRI), changes in blood markers, and behavioral changes in the subject (e.g., appetite, the ability to eat solid foods, grooming, sociability, energy levels, increased activity levels, weight gain, exhibition of increased comfort). These parameters can be measured using any methods known in the art. In varying embodiments, the different parameters can be assigned a score. Further, the scores of two or more parameters can be combined to provide an index for the subject.

Observation of the stabilization, improvement and/or reversal of one or more symptoms or parameters by a measurable amount indicates that the treatment or prevention regime is efficacious. Observation of the progression, increase or exacerbation of one or more symptoms indicates that the treatment or prevention regime is not efficacious. For example, in the case of cardiomyopathy, observation of the improvement of cardiac function (e.g., blood pressure in appropriate range, stable heart rhythm or reduction or absence of arrhythmias, changes in blood markers, and/or behavioral changes in the subject (e.g., increased appetite, the ability to eat solid foods, improved/increased grooming, improved/increased sociability, increased energy levels, improved/increased activity levels, weight gain and/or stabilization, exhibition of increased comfort) after one or more co-administrations of CBSCs with an agent indicates that the treatment or prevention regime is efficacious. Likewise, observation of reduction or decline of cardiac function (e.g., blood pressure in appropriate range, unstable heart rhythm or continued presence or increased arrhythmias, changes in blood markers, and/or behavioral changes in the subject (e.g., decreased appetite, the inability to eat solid foods, decreased grooming, decreased sociability, decreased energy levels, decreased activity levels, weight loss, exhibition of increased discomfort) after one or more co-administrations of CBSCs with an agent indicates that the treatment or prevention regime is not efficacious.

### Pharmaceutical Compositions

In certain embodiments, administration of any of the compositions embodied herein, can be combined with other cell-based therapies, for example, stem cells, TNFRII⁺ Treg cells, etc.

The compositions of the present disclosure may be prepared in a manner known in the art and are those suitable for parenteral administration to mammals, particularly humans, comprising a therapeutically effective amount of the composition alone, with one or more pharmaceutically acceptable carriers or diluents. The term “pharmaceutically acceptable carrier” as used herein means any suitable carriers, diluents or excipients. These include all aqueous and non-aqueous isotonic sterile injection solutions which may contain antioxidants, buffers and solutes, which render the composition isotonic with the blood of the intended recipient; aqueous and non-aqueous sterile suspensions, which may include suspending agents and thickening agents, dispersion media, antifungal and antibacterial agents, isotonic and absorption agents and the like. It will be understood that compositions of the disclosure may also include other supplementary physiologically active agents.

The carrier must be pharmaceutically “acceptable” in the sense of being compatible with the other ingredients of the composition and not injurious to the subject. Compositions include those suitable for parenteral administration, including subcutaneous, intramuscular, intravenous and intradermal administration. The compositions may conveniently be presented in unit dosage form and may be prepared by any method well known in the art of pharmacy. In general, the compositions are prepared by uniformly and intimately bringing into association any active ingredients with liquid carriers.

In certain embodiments, the composition is suitable for intramyocardial injection, parenteral administration. In another embodiment, the composition is suitable for intravenous administration. Compositions suitable for parenteral administration include aqueous and nonaqueous isotonic sterile injection solutions which may contain antioxidants, buffers, bactericides and solutes, which render the composition isotonic with the blood of the intended recipient; and aqueous and non-aqueous sterile suspensions which may include suspending agents and thickening agents.

In certain embodiments, the CBSC secretome compositions include pharmaceutically acceptable carriers. Additionally, in order to enhance the in vivo serum half-life of the administered compound e.g. osteoprotegrin (OPG) and/or insulin-like growth factor-binding protein 5 (IGFBP-5), the compositions may be encapsulated, introduced into the lumen of liposomes, prepared as a colloid, or other conventional techniques may be employed which provide an extended serum half-life of the compositions. A variety of methods are available for preparing liposomes, as described in, e.g., Szoka, et al., U.S. Pat. Nos. 4,235,871, 4,501,728 and 4,837,028 each of which is incorporated herein by reference. Furthermore, one may administer the drug in a targeted drug delivery system, for example, in a liposome coated with a tissue-specific antibody. The liposomes will be targeted to and taken up selectively by the organ.

The present disclosure also provides pharmaceutical compositions comprising one or more of the compositions described herein. Formulations may be employed in admixtures with conventional excipients, i.e., pharmaceutically acceptable organic or inorganic carrier substances suitable for administration to the wound or treatment site. The pharmaceutical compositions may be sterilized and if desired mixed with auxiliary agents, e.g., lubricants, preservatives, stabilizers, wetting agents, emulsifiers, salts for influencing osmotic pressure buffers, coloring, and/or aromatic substances and the like. They may also be combined, where desired, with other active agents, e.g., other analgesic agents.

Administration of the compositions of this disclosure may be carried out, for example, by parenteral, by intravenous, intratumoral, subcutaneous, intramuscular, or intraperitoneal injection, or by infusion or by any other acceptable systemic method. Formulations for administration of the compositions include those suitable for rectal, nasal, oral, topical (including buccal and sublingual), vaginal or parenteral (including subcutaneous, intramuscular, intravenous and intradermal) administration. The formulations may conveniently be presented in unit dosage form, e.g. tablets and sustained release capsules, and may be prepared by any methods well known in the art of pharmacy.

As used herein, “additional ingredients” include, but are not limited to, one or more of the following: excipients; surface active agents; dispersing agents; inert diluents; granulating and disintegrating agents; binding agents; lubricating agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the disclosure are known in the art and described, for example in Genaro, ed. (1985, Remington's Pharmaceutical Sciences, Mack Publishing Co., Easton, PA), which is incorporated herein by reference.

The composition of the disclosure may comprise a preservative from about 0.005% to 2.0% by total weight of the composition. The preservative is used to prevent spoilage in the case of exposure to contaminants in the environment. Examples of preservatives useful in accordance with the disclosure included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

### EXAMPLES

### Example 1: Cortical Bone Derived Stem Cells can Modulate Treg Plasticity and Phenotype to Orchestrate Myocardial Wound Healing Post Ischemic Injury

Myocardial Infarction (MI) solicits an inflammatory response that is closely coupled to cardiogenic wound healing process. T-regulatory cell (Treg), dynamics resident in the acute and chronically ischemic heart have proven to be instrumental in modulating wound healing processes post-MI. Cortical bone derived stem cells (CBSCs) can modulate the inflammatory microenvironment of the infarcted heart, but their exact mechanistic role in mediating adaptive immune response, specifically Treg phenotype and function, is unknown. It was hypothesized that CBSCs' cardiogenic capabilities lies in the interaction between cell signaling molecules retained in CBSC secretome with the adaptive inflammatory response mounted following ischemic injury.

### Materials and Methods

Cortical Bone Derived Stem Cell Isolation: Tibias and femurs were isolated from enhanced green fluorescent protein (eGFP)* C57BL/6J mice (2 to 6 months of age). Bone marrow was flushed from bone cavity and the remaining bone was digested using a collagenase-based digestion. Digested isolate was filtered, plated, sorted, and maintained in CBSC culture selection medium²⁵ until a homogenous population enriched in CBSC cell identification marker expression was achieved.¹⁹ CBSCs used in the present studies were between passages 15 and 20.

Mesenchymal Stem Cells Isolation: Tibias and femurs were isolated from C57BL/6J mice (2 to 6 months of age). Bone marrow was flushed from bone cavity with pre-warmed, complete culture medium containing DMEM (Corning, 10-013-CM), supplemented with 20% FBS (Neuromics, FBS0001-HI) and 1% Penicillin Streptomycin/L-glutamine (Gibco Life Technologies, 10378-016). Extracted marrow medium was filtered, purified, and cultured as previously described.^{19,26} MSCs used in present studies were between passages 3 and 4.

### Animal Procedures

I. Animal Procurement and Housing: All animal procedures and housing were approved by Temple University's Lewis Katz School of Medicine's Institutional Animal Care and Use Committee, an AAALAC accredited facility. C57BL/6J, C.Cg-Foxp3^{tm2Tch}/J, and B6.129(Cg)-Foxp3^{tm3(DTR/GFP)Ayr}/J animals were purchased from The Jackson Laboratory (Bar Harbor, ME).
II. Myocardial Infarction and Stem Cell Injection: For all animal procedures, mice (2-4 months of age) were anesthetized using 0.5-2% isoflurane. Permanent occlusion of the left anterior descending artery was occluded as previously described. 25.27 Heart pigmentation following occlusion was observed. Immediately following ligation, animals were injected with 100,000 CBSCs or MSCs suspended in sterile saline buffer at a total volume of 20 uL. Four, 5 uL intramyocardial injections (25,000 cells/injection) were placed along the infarct border zone of the LV. As a control, the same volume of sterile saline without stem cell resuspension was administered via intramyocardial injection. Animal breathing and temperature was monitored and controlled throughout procedure. Animals were also monitored for pain and distress during and following MI surgery.
III. Echocardiography: All animals were anesthetized using 0.5-2% isoflurane. Mice were placed on a heated elevated surface platform to maintain body temperature. Transthoracic echocardiography was performed using a Vevo2100 ultrasound system with MS400 transducer (VisualSonics; Toronto, Canada). B-Mode and M-Mode images were collected and analyzed using VevoLab v3.2.5 analysis software to determine EDV, ESV, FS, EF, LVPW, IVS, and LVID as previously described.28 Echocardiography assessment was determined at baseline, 1 week, and 8 weeks post-MI.
IV. FTY720 Administration: Female mice were injected (1 mg/kg) with reconstituted FTY720 (Sigma-Aldrich, SML0700). First dose was given 24 hours prior to MI surgery and daily for 1-week post-MI.
V. DTR Administration: Reconstituted DTR (Sigma-Aldrich, D0564) was administered (50 ng/mg) for 1 week prior to MI at. Last injection was administered the day prior to MI.

Immune Cell Isolation From Heart Tissue: Hearts were excised from the thoracic cavity animals anesthetized with 2.5-3% isoflurane 1 and 8 weeks post-MI. Hearts were trimmed, flushed, and weighed. The whole heart was minced and placed in digestive buffer (20 mM HEPES, 450U/mL Collagenase Type I (Worthington, LS004197), 60U/mL Deoxyribonuclease I (Worthington, LS002060), 60U/mL Hyaluronidase (Worthington, LS005474), and 125 U/mL Collagenase Type XI (Sigma, C7657) pHed to 7.4). The digestive buffer containing the minced heart was then placed on a rotating platform at 37° C. for 40 minutes. Cardiac Tissue debris was separated using gravimetric separation for 5 minutes. The remaining supernatant was collected and mixed with ice cold collagenase stopping buffer (10% FBS in PBS) and subsequently filtered through a 70 μM filtration column. Filtrate was centrifuged at 50g for 5 minutes to eliminate cardiomyocyte isolate. Remaining supernatant was collected and immune cells were further isolated via centrifugation at 500g at 4C. Pelleted cells were resuspended in red blood cell lysis buffer (155 mM NH₄Cl, 10 mM KHCO₃, 100 μM EDTA) pHed to 7.3 for 5 minutes. Remaining cellular isolate was pelleted and resuspended in cell stain buffer (4% FBS in PBS) and stained for T Cell identification markers to be assessed through flow cytometry as outlined below.

Immune Cell Isolation From Spleen: Spleens were excised from the body cavity of mice anesthetized with 2.5-3% isoflurane 1 and 8 weeks post-MI. Spleens were trimmed, rinsed with PBS, and weighed. Isolated spleens were placed on a 70 um mesh filter and mashed using the plunger of a sterile syringe. Immune cell isolation buffer (2% FBS+1 mM EDTA) pHed to 7.4 was passed through 70 μM filter. Immune Cells were pelleted by centrifugation 500g at 4° C. and resuspended in red blood cell lysis buffer, as outlined above. Remaining cellular isolate was re pelleted and resuspended in cell stain buffer. Cellular isolate was then pelleted and resuspended in cell stain buffer and stained for the T-cell Identification Markers and assessed via flow cytometry, as outlined below.

T Cell Population Characterization and Flow Cytometry: Immune cell isolates of the heart and spleen were stained for the following markers: Live/Dead (Aqua Zombie), CD45, CD3, CD4, CD8, CD25, FoxP3, TNFRI, and TNFRII. A list of antibodies and stain concentrations are supplied in Table 1, found below.

| Marker | Antibody Dilution (/100 uL) | | Company/Catalog# |
|---|---|---|---|
| Aqua Zombie | 0.13 | μL | Biolegend/423101 |
| CD45-PECy7 | 1.25 | μL | Invitrogen/25-0451-82 |
| CD3-BV605 | 1.25 | μL | Biolegend/100237 |
| CD4-PE | 1.25 | μL | Invitrogen/12-0041-82 |
| CD4-APC ef780 | 1.25 | μL | Invitrogen/47-0041-82 |
| CD8-APC ef780 | 1.25 | μL | Invitrogen/47-0041-82 |
| CD25-AF647 | 1.25 | μL | BD Pharmigen/563598 |
| FoxP3-ef450 | 0.5 | μL | Invitrogen/ 48-5773-82 |
| CD120a | 1.25 | μL | Biolegend/113003 |
| CD120b | 5 | μL | Biolegend/113405 |

In short, isolated immune cells were pelleted in 96 well v-bottom plates and were resuspended in aqua zombie viability dye in accordance with the manufacturer's protocol (Biolegend, 423101). For cell surface staining, cells were pelleted and resuspend in cell stain buffer containing antibodies against the following T Cell markers makers: CD45, CD3, CD4, CD8, CD25, CD120a, and CD120b at the concentrations listed in Table 1 for 20 minutes at ambient temperatures. Cells were subsequently fixed and permeabilized for Foxp3 staining using the True-Nuclear Fixation and Permeabilization kit in accordance with manufacturer's protocol (Biolegend, 424401). Following permeabilization, the FoxP3 antibody was diluted in permeabilization buffer and incubated with the immune cell suspension at ambient temperature for 20 minutes. Flow cytometry analysis of T cell population dynamics for the T cell markers outline above was acquired via the LSRII Flow Cytometer (BD) equipped with Diva digital acquisition software. Population dynamic percentages were quantified using Flow Jo v10 based on the gating strategies depicted in FIGS. 9A, 9B.

Mason Trichrome: At 1 week post-MI, hearts were excised, weighed, and perfused with PBS followed by formalin fixation. Hearts were routinely processed, embedded in paraffin, and sectioned for histochemistry. Masson's Trichrome staining was performed using Trichrome staining kit (Sigma-Aldrich, HT 15-1 KT) and Weigert's Iron Hematoxylin (Sigma Aldrich, HT1079-1SET) as per manufacturer's protocols.

RNA-Sequencing and Preparation: RNA sequencing data was collected as previously described.19 Raw fastq files were indexed to mouse genome mm 10 and quantified for downstream analysis by Salmon. Bioinformatic Analysis: Quantified transcripts were imported as a matrix of average transcript length using package tximport. to package DESeq2 for downstream differential expression analysis in R. Genes were identified and those with less than 5 reads per samples were removed. Wald test was used to statistically identify genes with P value<0.05. Using regularized logarithm (rlog) function, count data was transformed for visualization on a log 2 scale. Gene Ontology Analysis: Gene ontology (GO) enrichment analysis was performed using package goseq to classify genes in terms within biological process. Thresholds ofp>0.05 and 25% FDR were implemented.

T Cell Expansion Assays: T cells were isolated from FoxP3GFP mouse (2 to 6 months of age) using the immune cell isolation from spleen protocol outlined above and magnetically separated for CD4⁺ expression in accordance with manufacturer's protocol (Stem Cell Technologies, 19852A). CBSC pre secretome medium was collected and concentrated using Ultracentrifugation Systems (Millipore, UFC905024) and supplemented with 30U/mL of IL-2 (Peprotech,212-12). T cells (1×10⁶ cell/mL) were plated in CBSC pre-secretome medium or basal T cell medium (RPMI 1640 supplemented with 10% heat-inactivated FBS, 1% PSG, 10 mM HEPES, and 50 μM B-mercaptoethanol (Sigma-Aldrich, M3148) in CD3 (Invitrogen, 16-0032-85) and CD28 (Invitrogen, 16-0281-85) antibody (1 mg/mL) coated 96 well plates. Treg populations dynamics were assessed 4 days post, pre-secretome medium exposure via flow cytometry.

Treg Induction Assay: T cells were isolated from C57BL/6J (2 to 6 months of age) using the spleen immune cell isolation protocol outlined above. Immune cell isolate was collected and separated for CD4+CD25⁺ expression using a Treg magnetic cell isolation kit in accordance with manufacturer's protocol (Stem Cell Tech, 18783A). Once fully isolated, 80,000 T cells were isolated and resuspend in mediums previously exposed to CBSC or MSC populations, standardized to stem cell number. Basal medium consisted of DMEM medium (supplemented with 10% FBS and 1% PSG). Collected pre-secretome media were concentrated as outlined above and supplemented with 2000U/mL of IL-2. Cell cultures were plated in the presence of a 2:1 CD3/28 dynabead: cell ratio according to manufacturer's recommendations (Gibco Life Technologies, 11453D). Approximately 48 hours post-pre secretome medium exposure, T cells were isolated from culture and stained for the following Treg cell markers: CD4⁺, CD25⁺, and FoxP3⁺ using the cell staining protocol previously outlined above. Population dynamics were quantified via flow cytometry.

Edu Incorporation Assay: To assess Treg proliferation post stem cell pre secretome medium exposure, CD4⁺ CD25⁺ T cells were isolated from mouse spleen using Treg magnetic isolation kit, referenced above. Pure CD4⁺ CD25⁺ Treg cells were cultured in the presence of CBSC or MSC pre secretome mediums under the same conditions previously described above. CBSC and MSC pre secretome culture mediums were supplemented with 10 μM Edu (Invitrogen, C10337). Treg Edu incorporation was assessed at 48 hours post pre-secretome medium exposure via flow cytometry.

Cell Proliferation Dye Assay: To assess Treg proliferation via dye dilution, T cells were isolated from FoxP3GFP mouse (2 to 6 months of age) using the immune cell isolation from spleen protocol outlined above and separated for CD4⁺ expression as previously referenced. Isolated CD4+ T cells were stained with Far Red Cell Proliferation Dye in accordance with manufacturer's protocol (Invitrogen, C34572). CBSC and MSC pre-secretome medium, as outlined above, was supplemented with 30U/mL of IL-2 in the presence of a 1:1 CD3/28 dynabead: cell relationship. Dye dilution of far red proliferation dye was assessed 72 hours post, pre-secretome medium exposure via flow cytometry.

RNA Interference: CBSCs were transiently transfected in 6 well plates (50,000 cells/well) with 5 nM siOPG (Dharmacon, D-058801-01) using DharmaFECT 1 (Dharmacon, T-2001-03) transfection reagent (3 uL in 192 uL of serum free F12 medium) as per manufacturer's recommendations for 48 hours. Culture mediums were then replaced with T cell culture medium, outlined above, for 24 hours. T cell culture Medium was collected, concentrated, supplemented with 30U/mL of IL2, and exposed to T cell populations (1×10⁶ cells/mL) in 96 well plates coated with CD3 (and CD28 antibodies as outlined above. T cell populations were isolated from culture 4 days post-medium introduction and population dynamics were subsequently assessed via flow cytometry.

Lentiviral Knockdown of OPG: Recombinant lentivirus pLV[shRNA]-mCherry-U6>mTnfrsf11b[shRNA #1] (Vectorbuilder, LVS(VB210215-1129ujs)-C) that targets OPG expression and scramble control, recombinant lentivirus, pLV[shRNA]-EGFP/Puro-U6>Scramble_shRNA (Vectorbuilder, LVM(VB151023-10034)-C) were introduced to wtCBSC cell populations (MOI50) in 6 well plates (50,000 cells/well) containing 0.1% polybrene in complete CBSC culture medium. Medium was replaced 18 hours after exposure and cells were allowed to expand. Confirmation of transfection efficiency was assessed via flow cytometry 72 hours after transfection for mcherry” expression. The successful knockdown of OPG expression was assessed via RT-qPCR and western blot, using the protocols outlined below. OPG deficient cell lines were then incorporated into 1 week in vivo MI experimental design and heart and spleen immune cell composition 1 week post MI was assessed as outlined above.

### CBSC Secretome Quantification:

I. Paracrine Profiling: CBSC and MSC culture supernatants protein content was assessed and normalized using the Bicinchoninic Assay (Thermo Scientific, 23227) according to manufacturer's protocols. Equal amounts of protein were immunoblotted for 111 cytokines and chemokines using the Proteome Profiler Mouse XL Cytokine Array (R and D Systems) according to manufacturer's protocol with IRDye 800CW (1:2000, Li-Cor, 925-32230) for Li-Cor Imagining. Array was imaged using a Li-COR Odyssey Crx (Li-Cor Biosciences, Lincoln, NE). Densitometry analysis was performed using Image Studio (Li-Cor) analysis software.
II. Western Blot: Western Blot analysis was performed as described previously described.^{21,29} Briefly, sample protein concentrations were determined using the Bicinchoninic assay (BCA) in accordance with manufacturer's protocol. Protein samples were on a Mini-PROTEAN TGX Gels (Bio-rad,4561096). Primary antibodies against OPG (1:50, Abcam, ab5694) and GAPDH (1:500, Millipore Sigma, MAB374) were incubated overnight at 4° C. Secondary conjugated light-sensitive IRDye secondary antibodies were used to assess OPG (IRDye 800CW/1:1000, Li-Cor,926-32213) and GAPDH (IRDye 680RD/1:1000, Li-Cor,926-68072) expression. Secondary antibodies were incubated for 1 hr at room temperature and visualized using a Li-COR Odyssey Crx (Li-Cor Biosciences, Lincoln, NE). Densitometry analysis was performed using Image Studio (Li-Cor) analysis software.
III. Real Time Reverse Transcriptase-Polymerase Chain Reaction: To determine transcript expression of OPG in MSCs and CBSCs, total RNA was isolated from each stem cell using the RNeasy Kit (Qiagen) following the manufacturer's protocol. Single-Stranded cDNA was synthesized from 300 ng of RNA using the applied biosystem cDNA kit (Applied biosystems). Real Time Polymerase chain reactions were performed using CFX96 Real-Time System (Bio-Rad, CA) to acquire transcript amplification cycle numbers. Each PCR reaction contained of 0.15 μM Forward and Reverse Primers for OPG and 18s. The primers used to assess OPG content are presented in Table 2 found below.

Statistical Analysis: All data are expressed as a mean+SD. Comparison between multiple groups were done by 1—or 2-way ANOVA, a P<0.05 was statistically significant. Singular group comparisons were done via student's T-Test. P<0.05 was considered statistically significant. Statistical analysis was performed using GraphPad Prism version 8.0.

### Results

### CBSCs Establish a Pro-Reparative Inflammatory Response to Ischemic Injury

In previous reports, the inventors identified that CBSC cell therapy can alter the inflammatory microenvironment of the infarcted heart by increasing anti-inflammatory and reducing pro-inflammatory signaling molecule gene expression in the infarct border zone of the post-MI heart. However, whether the inflammatory cell infiltrate of the heart is altered in the presence of CBSC cell therapy was not assessed.^{19,20} Given CBSCs can sustain residence in the infarcted heart up to 14 days post-MI,¹⁹ the inflammatory cell infiltrate of the adaptive immune response was assessed 1 week post-MI via flow cytometry for the following expression T cell expression markers: CD45, CD3, CD4, CD8, CD25, and FoxP3 (FIG. 1A. and FIGS. 9A, 9B). Resident leukocyte populations were ˜3.5× and ˜2.5× greater in post-MI animals treated with CBSC cell therapy when compared to PBS (p<0.001) and MSC (p<0.001) treated controls, FIG. 1B. No significant alterations in global T cell population dynamics were observed, FIG. 1C. CD4⁺ T helper cell populations were expanded in CBSC treated animals by 12% when compared animals administered PBS injection (p<0.001), a trend not observed in MSC treated animals. CBSC cell therapy significantly expanded CD4⁺ resident T cell populations in the infarcted heart 1-week post-MI to a greater degree than their MSC counterparts (p<0.001), FIG. 1D. No global changes in CD8⁺ cytotoxic T cell compartments were observed, FIG. 1E. Further investigation into the expanded CD4⁺ T cell identified a 2 fold increase in Total and 2.5 fold increase in Total (p<0.001) and activated (p<0.0001) Treg compartments in CBSC treated animals compared to saline treated controls, FIGS. 1F and 1G. MSC cell therapy possessed increased trends of total and activated Treg content compared to PBS treated controls, but the expansion of Treg compartments were significantly less compared to CBSC treated animals (Total: 2.6%,p<0.01; Activated: 1.9%, p<0.001), FIGS. 1F and 1G.

Previous reports have identified variances in Treg population dynamics of the infarcted heart with TNFRI Tregs displaying a pathogenic Treg signature and TNFRII⁺ Tregs possessing pro-reparative signatures; therefore the population dynamics of the Treg compartments residing in the infarcted heart of CBSC treated animals was further investigated. Employing a flow cytometry gating scheme to assess TNFRI and TNFRII expression (FIG. 9B), it was identified that pathogenic, TNFRI⁺ Treg populations are significantly repressed by 10% in CBSC (p<0.0001) and MSC (p<0.0001) treated animals compared to PBS treated controls, FIG. 1H. No alterations in TNFRI⁺ Treg compartments were observed between MSC and CBSC treated animals. Pro-reparative TNFRII⁺ Treg signatures were increased by 12 and 19%, when compared to MSC (p<0.01) and PBS (p<0.0001) treated controls, respectively, FIG. 1I. CBSC cell therapy modulates the adaptive immune response to facilitate the expansion of pro-reparative TNFRII⁺ Treg populations in the infarcted myocardium 1—week post-MI, FIGS. 1A-1Q.

To determine if CBSC cell therapy modulates the systemic inflammatory response to MI, the inflammatory cell composition of the splenic tissue in animals CBSC, MSC, or PBS treated animals 1—week post-MI was investigated. Despite small increases in leukocyte (1.04×, p<0.05) and T-cell (1.14×, p<0.05) composition compared to PBS and MSC treated animals, respectively, no alterations in CD4⁺, CD8⁺, or T regulatory cell content was observed, FIGS. 1J-10. However, observations in TNFRI and TNFRII Treg signatures paralleled what was observed in the heart 1 week post MI. CBSC (p<0.0001) and MSC (P<0.0001) cell therapies significantly reduced pathogenic, TNFRI⁺ Treg compartments compared to PBS treated controls. CBSC treated animals also significantly expanding pro-reparative, TNFRII⁺ Treg compartment by 7.7% in MSC treated animals (p<0.01) and 4.4% in PBS (p<0.01) treated control, FIGS. 1P and 1Q. No alteration in spleen, liver, or kidney morphology was observed in CBSC, MSC, or PBS treated animals (FIGS. 12A-12J).

### CBSC Mediated Establishment of Tregs is Essential for Myocardial Wound Healing

To ensure the reparative mechanisms of CBSC cell therapy are in fact mediated through pro-reparative Treg expansion, two methods of Treg deletion were pursued. FoxP3^{DTR} animals were administered 50 ng/mg daily, 1 week prior to MI and CBSC cell therapy administration to deplete all FoxP3 expressing cells. Mason's Trichrome Staining identified Treg ablation causes an expansion in infarct size by 10.5% in CBSC treated animals and 4.5% in saline treated controls, FIGS. 2A and 2B. Removal of Treg cell populations from the infarcted heart greatly compromises the infarct limiting effects originally provided by CBSC cell therapy to a greater degree than PBS treated controls.

Previous reports identified 95% of the Treg compartment occupying the ischemic heart 1 week post-MI is recruited from systemic lymphoid stores.¹⁷ As a point of concept study, FTY720, an SIP1 receptor agonist, was administered to block Treg egression from systemic lymphoid stores and assess myocardial integrity and function via echocardiography 1 week post MI. CBSC and Saline treated animals treated with the SIP1 inhibitor FTY720 exhibited signatures of adverse cardiac remodeling marked by increase LV chamber volumes, wall thinning, and compromised cardiac function, FIGS. 2E-2M. Leukocyte, T-cell, CD4⁺, CD8⁺, and activated Treg content in the 1-week post-MI heart were significantly reduced in FTY720 animals compared to nontreated controls (FIGS. 2N-2R). Previous Treg expansion observed in non FTY720 treated controls, was normalized to Treg cell compartments of PBS treated control, further verifying CBSC cell therapy facilitates an important role in mediating the recruitment and expansion of Treg from peripheral T cell stores into the infarcted heart post-MI. The administration of FTY720 also affects T cell population dynamics in the spleen without affecting global systemic organ morphology of the spleen, kidney, and liver. (FIGS. 13A-13K) Interestingly, total and activated Treg cell compartments appear to be expanded in CBSC and PBS treated animals administered with FTY720, suggesting the spleen may serve as an induction site of Treg mediated activation and recruitment to the ischemic heart, FIGS. 13E and 13F).

### CBSC Cell Therapy Sustains Pro-Reparative Treg Cell Residence During Chronic Ischemic Injury

Previous reports identify Treg cells can diverge into pathogenic, TNFRI⁺ Treg populations during sustained, chronic inflammatory states.¹⁸ Ideally, the sustain of pro-reparative TNFRII⁺ Treg in the infarcted heart would prevent the divergence of pathogenic Treg populations and reduce adverse cardiac remodeling solicited by these pathogenic TNFRI⁺ expressing Treg cell types during chronic ischemic injury. Therefore, the same flow cytometry characterization panels were employed on the inflammatory cell infiltrate the chronically ischemic heart, 8 weeks post-MI, FIG. 3A. Resident leukocyte populations remained elevated in CBSC treated animals (14.7%) compared to MSC (7.2%,p<0.001) or PBS (4.8%,p<0.0001) treated controls, FIG. 3B. Global T Cell populations were reduced in MSC treated animals but remained sustained in CBSC (p<0.05) and PBS (p<0.05) treated animals, FIG. 3C. Unlike the data present at 1-week post-MI the CD4⁺ T cell compartment in CBSC treated animals is not expanded when compared to PBS and MSC treated controls, FIG. 3D. MSC treated animals show marked expansion of CD8⁺ cytotoxic T cell content (27%) verses CBSC (20.4%, p<0.01) or PBS (22.5%, p<0.05) treated counterparts, FIG. 3E. Further investigation into the expanded CD4⁺ T cell releveled a 1.5-fold increase in Total (p<0.001) and 2.3-fold increase in activated (p<0.0001) Treg compartments in CBSC treated animals compared to saline treated controls (FIGS. 3F and 3G). MSC cell therapy also slightly increases total and activated Treg content, but this expansion was significantly less compared to CBSC treated animals (Total: p<0.01; Activated: p<0.001), FIGS. 3F and 3G. Unlike 1-week post-MI, MSC therapy does not suppress pathogenic TNFRI+ Treg expression (15%) when compared to PBS treated animals (7%, p<0.001) or CBSC (2.89%, p<0.0001) treated animals. CBSCs significantly reduce pathogenic Treg populations compared to PBS (p<0.01) and MSC treated controls (p<000.1), FIG. 3H. In addition, CBSC cell therapy significantly expands pro-reparative TNFRII+ Treg populations by 2% and 3% in CBSC treated animals compared to PBS (p<0.05) and MSC (p<0.01) treated controls, respectively, FIG. 2I. CBSC cell therapy facilitates the direct expansion of pro-reparative Treg population during initial ischemic injury which is sustained during chronic stages of ischemic injury.

To determine if CBSC cell therapy modulates the systemic inflammatory response during chronic ischemic injury 8 weeks post-MI, the inflammatory cell composition of the spleen was also examined. At 8 weeks post MI, PBS (92.4%, p<0.05p) and CBSC (93.2%, p<0.01) treated animals had increased leukocyte compositions compared to MSC treated animals (88.9%), FIG. 2J. No alterations in global CD3⁺ T cell populations were observed across treatment groups, FIG. 3K. Both MSC (55.3%, p<0.001) and CBSC (51.6%, p<0.05) cell therapy contained expanded CD4⁺ compartments compared to PBS treated animals (49.6%), while PBS treated animals had greater cytotoxic CD8⁺ T cell compartments (42%) compared to MSC (38.2%, p<0.01) or CBSC (39.1%, p<0.01) treated animals, FIGS. 3L and 3M. Although no alteration in total Treg content was observed, activated Treg compartments are significantly expanded by 1.25 fold in MSC (p<0.05) and 1.3 fold in CBSC (p<0.01) treated animals when compared to saline treated controls, FIGS. 3N and 30. Further characterization of Treg compartment identifies that MSC therapy sustains a heightened pathogenic Treg signature (4.4%) in the spleen 8-week post-MI, whereas PBS (0.3%,p<0.05) and CBSC populations (0.08%,p<0.05) substantially diminish pathogenic TNFRI⁺ Treg signatures occupying the spleen, similar to the trend observed in the post-MI heart, FIG. 3P. CBSC cell therapy sustains the pro-reparative TNFRII⁺ Treg signature present in the spleen 1 week post-MI at 8 weeks post-MI by 6 and 5.5% in PBS (p<0.05) and MSC (p<0.05) treated animals, respectively, FIG. 3Q) CBSC cell therapy elicits systemic pro-reparative TNFRII* Treg expansion that parallels the pro-reparative TNFRII⁺ Treg populations observed in the infarcted heart during initial and chronic stages of cardiac injury.

CBSCs are Transcriptional Distinct from MSCs and Exhibit Enhanced Immunomodulatory Capabilities

CBSCs can directly modulate the adaptive immune response during initial and chronic stages of ischemic injury to a greater degree than other cell-based therapies, specifically MSCs, or saline treated animals; however, poor understanding surrounding the exact signaling factors produced by CBSCs is distinct from their closely residing MSC counterparts. Principle component analysis of CBSC and MSC transcriptomes identifies that CBSCs are transcriptional distinct populations compared to their MSC counterparts, FIG. 4A. The comparison of 13,146 genes between CBSC and MSC transcriptome identifies that nearly two thirds (57%) of the genes have significant (p<0.05) altered gene transcript expression. Approximately 3707 (28%) of the genes are significantly (p<0.05) downregulated, and 3827 (29%) are significantly (p<0.05) upregulated, FIGS. 4B and 4C. Gene ontology clustering of the 7,534 significantly altered gene transcripts, identifies 14 of the top 20 significantly altered gene ontology clusters are directly coupled to immune cell recruitment and function, chemotaxis, regulation of immune system processes, and cytokine secretion. The CBSCs transcriptome is enriched in immunomodulatory processes that make CBSCs functional distinct from their MSC counterparts and an ideal cell type to directly modulate the inflammatory microenvironment of the infarcted heart.

CBSCs possess a transcriptome enriched in processes that can directly modulate immune cell localization, function, and phenotype; therefore, the inventors set out to explore whether CBSC paracrine secretome can directly modulate Treg population dynamics. To determine whether CBSC secretome can facilitate Treg expansion, stem cell secretome medium previously exposed to CBSC populations was concentrated and exposed to CD4⁺ T cell cultures. Four days following pre-secretome medium exposure, Treg content was enriched by a 1.5 fold compared to CD4⁺ populations exposed to basal T cell growth culture mediums (p<0.0001), FIGS. 5A and 5B. Next, it was determined whether CBSC secretome could induced Treg cell formation from naïve, non-Treg expressing cell populations. Naïve CD4, CD25—T Cell populations were exposed to CBSC pre-secretome medium, which resulted in the expansion of Total (p<0.001) and Activated (p<0.001) Treg cell compartments by 15% and 13.5%, respectively, when compared to MSC pre-secretome mediums, FIGS. 5C-5F. CBSC pre secretome medium facilitates this expansion by soliciting Treg proliferation indicated by marked increases in Edu incorporation and cell membrane dye dilution, FIGS. 5G-5J. The secretome produced by transcriptionally distinct CBSC populations can solicit Treg induction, expansion, and proliferation in comparison to the extensively studied MSC cell line.

CBSC produced Osteoprotegrin is Essential for Pro-Reparative Treg Induction and Localization to the Infracted Heart

The secretome produce by CBSCs can directly modulate T cell response and Treg establishment. CBSC transcriptional expression of Osteoprotegrin (OPG) is increased by 3.5× and 46× in transcript and paracrine profiling of OPG, respectively, when compared to their MSC counterparts, FIGS. 6A and 6B. Consequently, it was investigated whether Treg population dynamics would be significantly altered when exposed to CBSC secretome deficient in OPG production. Small interfering RNAs targeting Tnfrsf11b were transfected into CBSC cultures for 48 hours and OPG knockdown was confirmed via RTqPCR and western blot, FIGS. 6A-6C. CBSC pre-secretome mediums were isolated from CBSCs deficient in OPG and were exposed to T cell populations. After 4 days, Treg cell induction and TNFRI/II expression was assessed. Total Treg establishment was repressed by 1.5× (p<0.0001) in T cells treated with CBSC medium deficient in OPG, FIGS. 6C and 6D. Further characterization of Treg population dynamics identifies a marked expansion (p<0.001) of pathogenic, TNFRI+ Treg populations exposed to OPG deficient CBSC secretome, FIGS. 6E and 6F. Additionally, pro-reparative, TNFRII⁺ Treg signatures are reduced (p<0.0001) when exposed to CBSC pre-secretome mediums deficient in OPG, FIGS. 6G and 6H. Together, this data demonstrated that OPG and IGFBP-5 are necessary for CBSC secretome mediated establishment of pro-reparative TNFRII⁺ Treg populations.

To further confirm the essential role of OPG in mediating pro-reparative Treg establishment in the infarcted heart, CBSCs were transfect with lentivirus target OPG expression or scramble control. Transfection efficiency and OPG knockdown were confirmed by mcherry expression via flow cytometry, RTqPCR, and western blot, FIGS. 14D-14H. Once OPG knockdown was confirmed, CBSCs deficient in OPG were incorporated into MI experimental design to assess the dependence of the pro-reparative Treg inflammatory response on CBSC mediated production of OPG, FIG. 7A. At 1 week following MI, the inflammatory microenvironment of the infarcted heart and the systemic splenic inflammatory profile was assessed via flow cytometry, FIGS. 9A and 9B. Hearts injected with CBSCs deficient in OPG production possessed marked reductions in global CD45⁺ Leukocytes (8%) and CD3⁺ T Cell (5.9%, p<0.05) content 1 week post MI, FIGS. 7B and 7C. Additionally, CD4⁺ T helper cell content was reduced by 6% (p<0.05), while CD8⁺ T cell content was not affected by CBSC cell therapy deficient in OPG expression, FIGS. 7D and 7E. Both Total (p<0.05) and Activated Treg (p<0.05) cell compartments were significantly reduced when compared to animals treated with CBSC scramble control, FIGS. 7F and 7J. Further investigation into Treg population dynamics were also altered when treated with OPG deficient CBSCs. Pathogenic, TNFRI⁺ Tregs occupying the ischemic heart were expanded by 1.3 fold, while pro-reparative TNFRII⁺ Tregs were reduced by 0.37 fold (p<0.01) in the infarcted heart 1 week post-MI, FIGS. 7H and 7I).

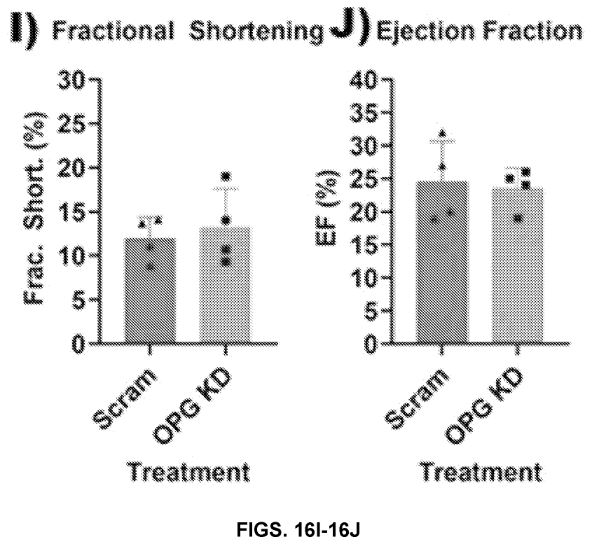

Quantification of immune cell occupancy in splenic tissue 1-week post-MI in MI animals treated with OPG deficient CBSCs did not possess alterations in global leukocyte, CD3⁺, CD4⁺ or CD8⁺ T cell content, FIGS. 7J-7M. However, reductions in Total (p<0.01) and Activated Treg (p<0.001) populations were observed in animals treated with OPG deficient CBSCs compared to scramble controls, FIGS. 7N and 70. Additionally, pathogenic TNFRI⁺ Tregs were expanded in OPG deficient CBSC treated animals while pro-reparative, TNFRII⁺ Treg occupancy decreased by 4.86% (p<0.05), FIGS. 7P and 7Q. The administration of CBSCs deficient in OPG solicits trends associated with adverse cardiac remodeling, increased cardiac hypertrophy, and compromised cardiac function, FIGS. 15A-15F and 16A-16J. Global cardiac hypertrophy determined through the comparison of the heart weight to tibial length was increased by 1.39× (p<0.001) in animals treated with OPG deficient CBSCs, which is not observed in global systemic organ morphology of the spleen, kidney, or liver, FIGS. 15A-15F. Animals treated with CBSCs lacking OPG production solicits alterations that contribute to adverse cardiac remodeling associated with increases in EDV, ESV, LVID, and slight decreases in LVPW and EF when compared to scramble CBSC treated controls 1-week post-MI, FIGS. 16A-16J.

### CBSC Produced Osteoprotegerin Influences T Cell Apoptosis and Survival Signaling

Previously the inventors reported that CBSC cell therapy can reduce CD45* apoptosis in the infarcted myocardium, but the exact signaling factors produced by CBSCs to modulate this response was poorly unknown. In the present study downstream TNFR signaling was investigated in CD4⁺ T cell populations exposed to CBSC pre secretome medium isolated from wtCBSC or CBSCs deficient in OPG. CD4⁺ T-cells exposed to CBSC pre secretome medium deficient in OPG production possess a 7× increase in Total Caspase 3 (p<0.01) and a 1.3× increase in Cleaved Caspase 3 (p<0.001) compared to CD4⁺ T cell populations exposed to precondition mediums isolated from wtCBSCs, FIGS. 8A-8D. Additionally, CD4⁺ T-cells exposed to CBSC pre secretome medium deficient in OPG production possesses a 5.5-fold increase in Total AKT (p<0.0001) and a 0.4 fold decrease in phosphorylated AKT (p<0.05) compared to CD4⁺ T cell populations exposed to precondition mediums isolated from wtCBSCs, FIGS. 8E-8G. Together this data provides evidence that CBSCs produced OPG is an essential regulator of downstream TNFR signaling to repress cellular death and promote T cell survival and function.

### DISCUSSION

Cardiac wound healing is closely coupled to the inflammatory response mounted following ischemic injury of the myocardium.⁹ Temporal regulation of the immune response to myocardial injury has proven to be essential, for the shortening or suppression of the acute inflammatory stage, or the prolonged sustainment of the initial inflammatory response during chronic injury can greatly hinder cardiac repair processes.⁸ Initial, pre-mature clinical observations in the field of cardiac immunology coupled the inflammatory response to heart failure pathology; thus, the primitive thought to administer immunosuppressive agents to temper the aggressive inflammatory response would improve patient outcomes following MI. However, the lack in clinical improvement of MI patients treated with broad immune suppressive agents emphasizes that the inflammatory response following MI is a sophisticated, interconnected, and multilayered immune response that cannot be simply modulated by pan immunosuppression of all inflammatory cell types. 30-34 In this study, the profound effects CBSC secretome has in modulating the T-cell inflammatory response post-ischemic injury was emphasized. Briefly, it was identified that the immunomodulatory capabilities of CBSC cell therapy is encompassed in three key domains: 1) The marked expansion of pro-reparative Treg cell residence in the infarcted myocardium during the critical transition between acute inflammation and the resolution and repair phases of cardiac injury, 2) CBSCs' blunt pathogenic, malfunctional remodeling of Treg populations previously observed during chronic ischemic injury (8 weeks post-MI), and 3) CBSCs' possess a paracrine signaling secretome that is enriched in potent cytokine and chemotactic signaling intermediates, specifically OPG, which can facilitate pro-reparative TNFRII+ Treg phenotype expression, and improved Treg dependent cardiac wound healing responses. Together, this report, emphasis the importance of harnessing next generation cell-based therapies to effectively manage the inflammatory microenvironment of the heart to improve cardiac wound healing post-ischemic injury.

Approximately one week following MI, the pro-inflammatory, degradative inflammatory state yields to the resolution and repair phases that are dominated by anti-inflammatory, pro-reparative immune cell populations that foster angiogenesis, scar formation, and inflammation resolution.⁸ One of these cell populations is the Treg cell. The exact role Treg cells serve in modulating cardiac repair post ischemic injury has come with great discrepancy and the ablation of this population greatly improves^{18,32,33,35,36} or compromises³⁷⁻³⁹ the ability of the myocardium to heal following ischemic injury. These heterogenic studies can simply be explained by the phenotypic plasticity that has plagued our understanding of Treg cell biology. Recent evidence has identified that Treg cell populations residing in the heart can diverge from pro-reparative TNFRII* expressing Treg populations to malfunctional, pro-inflammatory, pathogenic TNFRI⁺ expressing Treg populations during times of chronic ischemic injury.¹⁸ This Treg phenotypic conversion is directly coupled to adverse cardiac remodeling and function, which can be reversed during total Treg cell ablation and reestablishment between 4—and 8-weeks post-MI.¹⁸ In the present study, it was demonstrated that the intramyocardial injection of CBSC cell therapy along the infarct border zone of the ischemic heart facilitates the expansion of pro-reparative TFNRII⁺ Treg populations in the heart 1-week post-MI and limits TNFRI+ pathogenic Treg cell expression (FIGS. 1A-1Q). Treg ablation in CBSC treated animals diminishes the reparative effects solicited by CBSCs marked by infarct size expansion and compromised cardiac function (FIGS. 2A-2R). Additionally, pro-reparative TNFRII⁺ Treg cell signatures are sustained 8 weeks post-MI and are coupled to improved cardiac function capabilities, replacing the need for total Treg ablation to prevent pathogenic Treg remodeling (FIGS. 3A-3Q).

The efficacy of cell-based therapy implementation to heal the infarcted heart via the direct differentiation of progenitor cell populations into new, functional myocardium has been relatively modest and is not considered the main cardiogenic role of cell-based therapies.¹ However, an increased emphasis on next generation cell-based therapy, in particular the paracrine secretome produced by progenitor cell populations and the direct role these secretomes have in modulating cardiac wound healing is the epicenter of the reparative mechanisms that surround the angiogenic,²⁵ fibroblast maturative,²¹ and immunomodulatory properties possessed by stem cell therapy.^{4,6} The CBSC secretome is enriched in paracrine signaling modalities that are closely coupled to the inflammatory response and cardiac wound healing processes (FIGS. 4A-4D). In the present study, it was identified that the CBSC secretome can directly mediate the induction, phenotype, and proliferation of Treg cells (FIGS. 5A-5J). Previously the inventors have shown CBSCs can expand pro-reparative inflammatory cell types in the infarcted myocardium 7 days post ischemic injury, but the exact signaling processes that mediate this establishment of the anti-inflammatory, pro-reparative microenvironment present during CBSC cell therapy administration is unknown.^{19,20} In the present study, the knowledge of the CBSC secretome was expanded by identifying CBSCs' enriched expression of OPG, a unique signature of the hard bone derived progenitor cell population when compared to their closely residing bone marrow derived MSC counterparts (FIGS. 6A-6H).

Osteoprotegerin has previously been reported to act as a decoy receptor that sequesters TNFR mediated signaling molecules, specifically RANKL, TRAIL, and sTNFa from binding to TNF receptors, all of which significantly modulate apoptotic induced cellular death and pro-survival AKT signaling.⁴⁰⁻⁴⁴ Osteoprotegerin has also been shown to modulate CVD pathology, as increases in OPG levels have been shown to be positively correlated with improved myocardial integrity in the ischemic and failing heart.⁴⁵⁻⁴⁸ In this report, it was demonstrated that CBSCs deficient in OPG production lose their ability to induce and localize pro-reparative, TNFRII⁺ Treg cells in the infarcted heart and compromise T cell survival signaling and increased incidence of apoptotic cell death (FIGS. 7A-7Q and 8A-8G).

In general, the understanding of Treg cell biology has significantly expanded throughout the last decade, leading to the discovery of Treg cell populations that facilitate roles that are extrinsic to the traditional roles they serve in modulating peripheral immune tolerance. Tissue Tregs (tsTregs) have been shown to be responsible for maintaining the homeostatic microenvironment of the tissues they populate, for the depletion of these populations significantly compromises tissue function and homeostasis. 16.49 Most recently, a Treg population that specifically populates the heart during ischemic injury has demonstrated to be necessary for mediating cardiac repair post-MI. This cardiac tsTreg population is transcriptionally distinct from other Treg cell populations that populate other organ structures and conventional lymphoid stores. 17 These cardiac specific Tregs appear to be mainly recruited from systemic lymphoid stores prior to their egression to the ischemic heart upon injury onset. The studies herein identified that CBSCs produce outward signaling that promotes the expansion of pro-reparative, TNFRII+ populations in the spleen, a systemic lymphoid organ. Other tsTreg cell populations, specifically Tregs that populate the visceral adipose tissue (VAT), appear to undergo transcriptional alterations that permit the adoption of the VAT Treg signature prior to their egression to the metabolically indecent VAT.⁵⁰

In summary, it was identified herein that CBSC cell therapy as superior modulator of Treg population dynamics in the infarcted heart. CBSC cell therapy is enriched in chemotactic signaling molecules that can modulate the entire inflammatory response to ischemic injury, but here a thorough investigation was provided into how CBSC produce OPG can solicit pro-reparative Treg establishment and sustainment in the heart during the acute and chronic stages of ischemic injury to better orchestrate myocardial wound healing.

### REFERENCES

1. Banerjee Monisha N., Bolli Roberto, Hare Joshua M. Clinical Studies of Cell Therapy in Cardiovascular Medicine. Circulation Research. 2018; 123:266-287.
2. Karantalis Vasileios, DiFede Darcy L., Gerstenblith Gary, Pham Si, Symes James, Zambrano Juan Pablo, Fishman Joel, Pattany Pradip, McNiece Ian, Conte John, Schulman Steven, Wu Katherine, Shah Ashish, Breton Elayne, Davis-Sproul Janice, Schwarz Richard, Feigenbaum Gary, Mushtaq Muzammil, Suncion Viky Y., Lardo Albert C., Borrello Ivan, Mendizabal Adam, Karas Tomer Z., Byrnes John, Lowery Maureen, Heldman Alan W., Hare Joshua M. Autologous Mesenchymal Stem Cells Produce Concordant Improvements in Regional Function, Tissue Perfusion, and Fibrotic Burden When Administered to Patients Undergoing Coronary Artery Bypass Grafting. Circulation Research. 2014; 114:1302-1310.
3. Makkar R R, Smith R R, Cheng K, Malliaras K, Thomson L E, Berman D, Czer L S, Marbán L, Mendizabal A, Johnston P V, Russell S D, Schuleri K H, Lardo A C, Gerstenblith G, Marbán E. Intracoronary cardiosphere-derived cells for heart regeneration after myocardial infarction (CADUCEUS): a prospective, randomised phase 1 trial. Lancet. 2012; 379:895-904.
4. Wagner M J, Khan M, Mohsin S. Healing the Broken Heart; The Immunomodulatory Effects of Stem Cell Therapy. Front Immunol [Internet]. 2020 [cited 2020 Oct 23]; 11. Available from: https://www.frontiersin.org/articles/10.3389/fimmu.2020.00639/full
5. Kraus L, Mohsin S. Role of Stem Cell-Derived Microvesicles in Cardiovascular Disease. J Cardiovasc Pharmacol. 2020; 76:650-657.
6. Mohsin S, Houser SR. Cortical Bone Derived Stem Cells for Cardiac Wound Healing. Korean Circulation Journal. 2019; 49:314-325.
7. Frangogiannis N G. The inflammatory response in myocardial injury, repair, and remodelling. Nat Rev Cardiol. 2014; 11:255-265.
8. Prabhu Sumanth D., Frangogiannis Nikolaos G. The Biological Basis for Cardiac Repair After Myocardial Infarction. Circulation Research. 2016; 119:91-112.
9. Sattler S, Kennedy-Lydon T, editors. The Immunology of Cardiovascular Homeostasis and Pathology [Internet]. Springer International Publishing; 2017 [cited 2019 Dec 4]. Available from: https://www.springer.com/gp/book/9783319576114
10. Malissen B, Bongrand P. Early T Cell Activation: Integrating Biochemical, Structural, and Biophysical Cues. Annu Rev Immunol. 2015; 33:539-561.
11. Rossjohn J, Gras S, Miles J J, Turner S J, Godfrey D I, Mccluskey J. T Cell Antigen Receptor Recognition of Antigen-Presenting Molecules. Annu Rev Immunol. 2015; 33:169-200.
12. Lu L, Barbi J, Pan F. The regulation of immune tolerance by FOXP3. Nature Reviews Immunology. 2017; 17:703-717.
13. Bennett C L, Christie J, Ramsdell F, Brunkow M E, Ferguson P J, Whitesell L, Kelly T E, Saulsbury F T, Chance P F, Ochs H D. The immune dysregulation, polyendocrinopathy, enteropathy, X-linked syndrome (IPEX) is caused by mutations of FOXP3. Nat Genet. 2001; 27:20-21.
14. Liston A, Nutsch K M, Farr A G, Lund J M, Rasmussen J P, Koni P A, Rudensky A Y. Differentiation of regulatory Foxp3⁺ T cells in the thymic cortex. PNAS. 2008; 105:11903-11908.
15. Sakaguchi S, Sakaguchi N, Asano M, Itoh M, Toda M. Immunologic self-tolerance maintained by activated T cells expressing IL-2 receptor alpha-chains (CD25). Breakdown of a single mechanism of self-tolerance causes various autoimmune diseases. J Immunol. 1995; 155:1151-1164.
16. Panduro M, Benoist C, Mathis D. Tissue Tregs. Annual Review of Immunology. 2016; 34:609-633.
17. Xia Ni, Lu Yuzhi, Gu Muyang, Li Nana, Liu Meilin, Jiao Jiao, Zhu Zhengfeng, Li Jingyong, Li Dan, Tang Tingting, Lv Bingjie, Nie Shaofang, Zhang Min, Liao Mengyang, Liao Yuhua, Yang Xiangping, Cheng Xiang. A Unique Population of Regulatory T Cells in Heart Potentiates Cardiac Protection From Myocardial Infarction. Circulation. 2020; 142:1956-1973.
18. Bansal S S, Ismahil M A, Goel M, Zhou G, Rokosh G, Hamid T, Prabhu S D. Dysfunctional and Proinflammatory Regulatory T-Lymphocytes Are Essential for Adverse Cardiac Remodeling in Ischemic Cardiomyopathy. Circulation. 2019; 139:206-221.
19. Mohsin S, Troupes C D, Starosta T, Sharp T E, Agra E J, Smith S, Duran J M, Zalavadia N, Zhou Y, Kubo H, Berretta R M, Houser S R. Unique Features of Cortical Bone Stem Cells Associated With Repair of the Injured Heart. Circulation Research. 2015; 117:1024-1033.
20. Hobby ARH, Sharp T E, Berretta R M, Borghetti G, Feldsott E, Mohsin S, Houser SR. Cortical bone-derived stem cell therapy reduces apoptosis after myocardial infarction. Am J Physiol Heart Circ Physiol. 2019;317:H820-H829.

21 Kraus L, Ma L, Yang Y, Nguyen F, Hoy R C, Okuno T, Khan M, Mohsin S. Cortical Bone Derived Stem Cells Modulate Cardiac Fibroblast Response via miR-18a in the Heart After Injury. Front Cell Dev Biol. 2020; 8:494.
22. Nakagawa N, Kinosaki M, Yamaguchi K, Shima N, Yasuda H, Yano K, Morinaga T, Higashio K. RANK Is the Essential Signaling Receptor for Osteoclast Differentiation Factor in Osteoclastogenesis. Biochemical and Biophysical Research Communications. 1998; 253:395-400.
23. Walsh MC, Choi Y. Biology of the RANKL-RANK-OPG System in Immunity, Bone, and Beyond. Front Immunol [Internet]. 2014 [cited 2021 Apr 19];5. Available from: https://www.frontiersin.org/articles/10.3389/fimmu.2014.00511/full
24. Weitzmann M N. The Role of Inflammatory Cytokines, the RANKL/OPG Axis, and the Immunoskeletal Interface in Physiological Bone Turnover and Osteoporosis. Scientifica (Cairo). 2013; 2013:125705.
25. Duran J M, Makarewich C A, Sharp T E, Starosta T, Zhu F, Hoffman N E, Chiba Y, Madesh M, Berretta R M, Kubo H, Houser S R. Bone-derived stem cells repair the heart after myocardial infarction through transdifferentiation and paracrine signaling mechanisms. Circulation Research. 2013; 113:539-552.

26 Williams Adam R., Hatzistergos Konstantinos E., Addicott Benjamin, McCall Fred, Carvalho Decio, Suncion Viky, Morales Azorides R., Da Silva Jose, Sussman Mark A., Heldman Alan W., Hare Joshua M. Enhanced Effect of Combining Human Cardiac Stem Cells and Bone Marrow Mesenchymal Stem Cells to Reduce Infarct Size and to Restore Cardiac Function After Myocardial Infarction. Circulation. 2013; 127:213-223.
27. Zhang Jianhua, Wilson Gisela F., Soerens Andrew G., Koonce Chad H., Yu Junying, Palecek Sean P., Thomson James A., Kamp Timothy J. Functional Cardiomyocytes Derived From Human Induced Pluripotent Stem Cells. Circulation Research. 2009; 104:e30-e41.
28. Yang Y, Schena G J, Wang T, Houser S R. Postsurgery echocardiography can predict the amount of ischemia-reperfusion injury and the resultant scar size. APSselect. 2020;8:H690-H698.
29. Borden Austin, Kurian Justin, Nickoloff Emily, Yang Yijun, Troupes Constantine D., Ibetti Jessica, Lucchese Anna Maria, Gao Erhe, Mohsin Sadia, Koch Walter J., Houser Steven R., Kishore Raj, Khan Mohsin. Transient Introduction of miR-294 in the Heart Promotes Cardiomyocyte Cell Cycle Reentry After Injury. Circulation Research. 2019; 125:14-25.
30 Huang S, Frangogiannis N G. Anti-inflammatory therapies in myocardial infarction: failures, hopes and challenges. Br J Pharmacol. 2018; 175:1377-1400.
31. Dobaczewski M, Xia Y, Bujak M, Gonzalez-Quesada C, Frangogiannis N G. CCR5 Signaling Suppresses Inflammation and Reduces Adverse Remodeling of the Infarcted Heart, Mediating Recruitment of Regulatory T Cells. The American Journal of Pathology. 2010; 176:2177-2187.
32. Weirather J, Hofmann U D W, Beyersdorf N, Ramos GC, Vogel B, Frey A, Ertl G, Kerkau T, Frantz S. Foxp3+ CD4⁺ T cells improve healing after myocardial infarction by modulating monocyte/macrophage differentiation. Circulation Research. 2014; 115:55-67.

33 Saxena A, Dobaczewski M, Rai V, Haque Z, Chen W, Li N, Frangogiannis N G. Regulatory T cells are recruited in the infarcted mouse myocardium and may modulate fibroblast phenotype and function. American Journal of Physiology-Heart and Circulatory Physiology. 2014;307:H1233-H1242.
34. Anzai Atsushi, Anzai Toshihisa, Nagai Shigenori, Maekawa Yuichiro, Naito Kotaro, Kaneko Hidehiro, Sugano Yasuo, Takahashi Toshiyuki, Abe Hitoshi, Mochizuki Satsuki, Sano Motoaki, Yoshikawa Tsutomu, Okada Yasunori, Koyasu Shigeo, Ogawa Satoshi, Fukuda Keiichi. Regulatory Role of Dendritic Cells in Postinfarction Healing and Left Ventricular Remodeling. Circulation. 2012; 125:1234-1245.
35. Hofmann U, Beyersdorf N, Weirather J, Podolskaya A, Bauersachs J, Ertl G, Kerkau T, Frantz S. Activation of CD4⁺ T Lymphocytes Improves Wound Healing and Survival After Experimental Myocardial Infarction in MiceClinical Perspective. Circulation. 2012; 125:1652-1663.
36. Tang T-T, Yuan J, Zhu Z-F, Zhang W-C, Xiao H, Xia N, Yan X-X, Nie S-F, Liu J, Zhou S-F, Li J-J, Yao R, Liao M-Y, Tu X, Liao Y-H, Cheng X. Regulatory T cells ameliorate cardiac remodeling after myocardial infarction. Basic Research in Cardiology. 2012; 107:232.
37. Sharir R, Semo J, Shimoni S, Ben-Mordechai T, Landa-Rouben N, Maysel-Auslender S, Shaish A, Entin-Meer M, Keren G, George J. Experimental Myocardial Infarction Induces Altered Regulatory T Cell Hemostasis, and Adoptive Transfer Attenuates Subsequent Remodeling. PLOS ONE. 2014;9:e113653.
38. Mathes D, Weirather J, Nordbeck P, Arias-Loza A-P, Burkard M, Pachel C, Kerkau T, Beyersdorf N, Frantz S, Hofmann U. CD4+ Foxp3⁺ T-cells contribute to myocardial ischemia-reperfusion injury. Journal of Molecular and Cellular Cardiology. 2016; 101:99-105.
39 Bansal S S, Ismahil M A, Goel M, Patel B, Hamid T, Rokosh G, Prabhu S D. Activated T Lymphocytes are Essential Drivers of Pathological Remodeling in Ischemic Heart Failure. Circ Heart Fail. 2017; 10:e003688.
40. Wajant H. Principles of antibody-mediated TNF receptor activation. Cell Death & Differentiation. 2015; 22:1727-1741.
41. Rochette L, Meloux A, Rigal E, Zeller M, Cottin Y, Vergely C. The Role of Osteoprotegerin and Its Ligands in Vascular Function. Int J Mol Sci. 2019;20.
42. Schoppet Michael, Preissner Klaus T., Hofbauer Lorenz C. RANK Ligand and Osteoprotegerin. Arteriosclerosis, Thrombosis, and Vascular Biology. 2002; 22:549-553.
43. Bucay N, Sarosi I, Dunstan C R, Morony S, Tarpley J, Capparelli C, Scully S, Tan HL, Xu W, Lacey D L, Boyle W J, Simonet W S. osteoprotegerin-deficient mice develop early onset osteoporosis and arterial calcification. Genes Dev. 1998; 12:1260-1268.
44 Hofbauer L C, Shui C, Riggs B L, Dunstan C R, Spelsberg T C, O'Brien T, Khosla S. Effects of immunosuppressants on receptor activator of NF-kappaB ligand and osteoprotegerin production by human osteoblastic and coronary artery smooth muscle cells. Biochem Biophys Res Commun. 2001; 280:334-339.
45. Omland T, Drazner M H, Ueland T, Abedin M, Murphy S A, Aukrust P, de Lemos J A. Plasma osteoprotegerin levels in the general population: relation to indices of left ventricular structure and function. Hypertension. 2007; 49:1392-1398.
46. Omland T, Ueland T, Jansson A M, Persson A, Karlsson T, Smith C, Herlitz J, Aukrust P, Hartford M, Caidahl K. Circulating osteoprotegerin levels and long-term prognosis in patients with acute coronary syndromes. J Am Coll Cardiol. 2008; 51:627-633.
47. Xi L, Cao H, Zhu J, Røe OD, Li M, Wu Y, Wang D, Chen Y. OPG/RANK/RANKL axis in stabilization of spontaneously restored sinus rhythm in permanent atrial fibrillation patients after mitral valve surgery. Cardiology. 2013; 124:18-24.
48. Shaker O G, El-Shehaby A, Nabih M. Possible role of osteoprotegerin and tumor necrosis factor-related apoptosis-inducing ligand as markers of plaque instability in coronary artery disease. Angiology. 2010; 61:756-762.
49. Lui PP, Cho I, Ali N. Tissue regulatory T cells. Immunology. 2020; 161:4-17.
50 Li C, DiSpirito J R, Zemmour D, Spallanzani R G, Kuswanto W, Benoist C, Mathis D. TCR Transgenic Mice Reveal Stepwise, Multi-site Acquisition of the Distinctive Fat-Treg Phenotype. Cell. 2018; 174:285-299.e12.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed:

1. A method of promoting myocardial homeostasis and cardiac wound healing in a subject in need thereof, comprising:
administering to the subject a therapeutically effective amount of cortical bone derived stem cells (CBSCs); thereby,
promoting myocardial homeostasis and cardiac wound healing in a subject

2. The method of claim 1, wherein the CBSCs are administered via intramyocardial injection.

3. The method of claim 1, wherein the CBSCs are autologous, allogeneic, haplotype matched, haplotype mismatched, haplo-identical, xenogeneic or combinations thereof.

4. The method of claim 1, further comprising administering a therapeutically effective amount of a CBSC secretome.

5. The method of claim 4, wherein the CBSC secretome comprises osteoprotegrin (OPG) and/or insulin-like growth factor-binding protein 5 (IGFBP-5).

6. The method of any one of claims 1-5, wherein the CBSCs and/or secretome induce T regulatory (Treg) cells.

7. The method of any one of claims 1-5, wherein the CBSCs and/or secretome mediate recruitment and expansion of Treg from peripheral T cell stores into the subject's infarcted heart.

8. The method of any one of claims 1-5, wherein the CBSCs and/or secretome modulate the inflammatory microenvironment of the subject's infarcted heart.

9. The method of any one of claims 1-8, wherein the induced Treg cells are TNFRII+.

10. The method of claim 1, wherein the CBSCs are expanded ex vivo.

11. The method of any one of claims 1-10, wherein the CBSC secretome is enriched ex vivo by culturing the CBSCs for at least 12 hours.

12. The method of any one of claims 1-11, further comprising culturing Treg cells ex vivo with the CBSC secretome.

13. The method of claim 12, wherein the Treg cells are TNFRII+.

14. The method of claim 13, wherein the TNFRII+ Treg cells are adoptively transferred to the subject.

15. A composition comprising a therapeutically effective amount of cortical bone derived stem cells (CBSCs).

16. The composition of claim 15, wherein the CBSCs comprise an expression vector encoding osteoprotegrin (OPG) and/or insulin-like growth factor-binding protein 5 (IGFBP-5).

17. A method of modulating T regulatory (Treg) cells in vivo, comprising administering to a subject a therapeutically effective amount of cortical bone derived stem cells (CBSCs) and/or CBSC secretome and/or CBSCs comprising an expression vector encoding osteoprotegrin (OPG) and/or insulin-like growth factor-binding protein 5 (IGFBP-5).
